(19) United States
(12) Patent Application Publication
(10) Pub. No.: US 2008/0152540 A1
(43) Pub. Date: Jun. 26, 2008

(54) PACKAGING SOLUTIONS

(75) Inventors: Derek A. Schorzman, Cary, NC (US); Richard I. Blackwell, Webster, NY (US); Jay F. Kunzler, Canandaigua, NY (US)

Correspondence Address: Bausch & Lomb Incorporated, One Bausch & Lomb Place, Rochester, NY, 14604-2701, US

(73) Assignee: BAUSCH & LOMB INCORPORATED, Rochester, NY (US)

(21) Appl. No.: 11/615,217

(22) Filed: Dec. 22, 2006

(51) Int. Cl. A61K 8/89; B01J 19/00; C08G 63/91

(52) U.S. Cl. 422/40; 525/54.2; 424/70.12

(57) ABSTRACT

The present invention is directed to new and improved packaging systems for storing cationic ophthalmic devices such as cationic contact lenses and to methods for packaging such cationic ophthalmic devices with solutions to improve the comfort of the lenses during wear. In particular, the present invention is directed to a packaging system for storing a cationic ophthalmic device in a solution comprising an effective amount of an anionic polymer. Such solutions are retained on the surface of an unused lens for extended periods of time, resulting in surface modification that persists in the eye, which may provide significant improvement in the wetting properties of fresh lenses used for the first time and, moreover, even several hours after lens insertion, preventing dryness and improving lubricity.

### BACKGROUND OF THE INVENTION

1. Technical Field

The present invention is directed to a new and improved packaging system for storing cationic ophthalmic devices such as cationic contact lenses.

2. Description of Related Art

Blister-packs and glass vials are typically used to individually package each soft contact lens for sale to a customer. Saline or deionized water is commonly used to store the lens in the blister-packs, as mentioned in various patents related to the packaging or manufacturing of contact lenses. Because lens material may tend to stick to itself and to the lens package, packaging solutions for blister-packs have sometimes been formulated to reduce or eliminate lens folding and sticking. For this reason, polyvinyl alcohol (PVA) has been used in contact-lens packaging solutions.

It has been stated that if a lens is thoroughly cleaned before insertion, lacrimal fluid can adequately wet the lens. Furthermore, the difficulties of adding a surfactant to a packaging solution, including the possibility of lowering shelf-life and/or adverse reactions during heat sterilization, have further limited the use of surfactants in a packaging solution for the purpose of providing any possible or marginal effect on lens comfort. It is only after a lens has been worn, when proteins or other deposits have formed on the surface of the lens, that surfactants have been used in standard lens-care solutions.

It is highly desirable that contact lens be as comfortable as possible for wearers. Manufacturers of contact lenses are continually working to improve the comfort of the lenses. Nevertheless, many people who wear contact lenses still experience dryness or eye irritation throughout the day and particularly towards the end of the day. An insufficiently wetted lens at any point in time will cause significant discomfort to the lens wearer. Although wetting drops can be used as needed to alleviate such discomfort, it would certainly be desirable if such discomfort did not arise in the first place.

U.S. Pat. No. 4,321,261 (“the '261 patent”) discloses a method of rendering a contact lens that has an ionic surface more compatible with the eye by immersing the lens in a solution of an oppositely charged ionic polymer to form a thin polyelectrolyte complex on the lens surface, which complex increases its hydrophilic character for a greater period of time relative to an untreated surface and which reduces the tendency for mucoproteins, a normal constituent of lacrimal tears, to adhere to a lens surface. However, there is no disclosure in the '261 patent of a packaging system.

It would be desirable to provide an improved packaging system for cationic ophthalmic devices such as a cationic contact lenses such that the lens would be comfortable to wear in actual use and would allow for the extended wear of the lens without irritation or other adverse effects to the cornea.

### SUMMARY OF THE INVENTION

In accordance with one embodiment of the present invention, a method of preparing a package comprising a storable, sterile cationic ophthalmic device is provided comprising:

(a) immersing an ophthalmic device having at least one cationic surface in a solution comprising an effective amount of a soluble anionic polymer, wherein the solution has an osmolality of at least about 200 mOsm/kg and a pH in the range of about 6 to about 9;

(b) packaging the solution and the lens in a manner preventing contamination of the lens by microorganisms; and

(c) sterilizing the packaged solution and device.

In accordance with a second embodiment of the present invention, a method for packaging and storing a cationic ophthalmic lens is provided comprising, prior to delivery of the ophthalmic lens to the customer-wearer, immersing the cationic ophthalmic lens in an aqueous packaging solution inside a package and heat sterilizing the solution, wherein the aqueous packaging solution comprises a sterile ophthalmically safe aqueous solution comprising an effective amount of a soluble anionic polymer, wherein the solution has an osmolality of at least about 200 mOsm/kg and a pH of about 6 to about 9.

In accordance with a third embodiment of the present invention, a packaging system for the storage of a cationic ophthalmic device is provided comprising a sealed container containing one or more unused cationic ophthalmic devices immersed in an aqueous packaging solution comprising an effective amount of a soluble anionic polymer, wherein the solution has an osmolality of at least about 200 mOsm/kg, a pH of about 6 to about 9 and is heat sterilized.

In accordance with a fourth embodiment of the present invention, a packaging system for the storage of a cationic ophthalmic lens is provided comprising:

(a) a solution comprising an effective amount of an anionic polymer, wherein the solution has an osmolality of at least about 200 mOsm/kg and a pH in the range of about 6 to about 9;

(b) at least one cationic ophthalmic lens; and

(c) a container for holding the solution and cationic ophthalmic lens sufficient to preserve the sterility of the solution and cationic ophthalmic device, wherein the solution does not contain an effective disinfecting amount of a disinfecting agent.

### DEFINITIONS

The term “monomer” and like terms as used herein denote relatively low molecular weight compounds that are polymerizable by, for example, free radical polymerization, as well as higher molecular weight compounds also referred to as “prepolymers”, “macromonomers”, and related terms.

The term “cationic monomer”, as used herein, refers to a monomer having a group in the chain of the monomer or a pendent functional group that exhibits a cationic (positive) charge either permanently or through protonation in water at physiological pH values, i.e., a pH of about 7.2 to about 7.

### BRIEF DESCRIPTION OF THE DRAWINGS

FIG. 1 is a graphical illustration of a normalized static co-efficient of friction (COF) values for a cationic contact lens in various packaging solutions of the present invention.

FIG. 2 is a graphical illustration of a normalized kinetic COF values for a cationic contact lens in various packaging solutions of the present invention.

### DETAILED DESCRIPTION OF THE PREFERRED EMBODIMENTS

The present invention provides a packaging system for the storage of a cationic ophthalmic device intended for direct contact with body tissue or body fluid in a solution containing an effective amount of an anionic polymer. As used herein, the term “ophthalmic device” refers to devices that reside in or on the eye. These devices can provide optical correction, wound care, drug delivery, diagnostic functionality or cosmetic enhancement or effect or a combination of these properties. Representative examples of such devices include, but are not limited to, soft contact lenses, e.g., soft, hydrogel lens; soft, non-hydrogel lens and the like, hard contact lenses, e.g., hard, gas permeable lens materials and the like, intraocular lenses, overlay lenses, ocular inserts, optical inserts and the like. As is understood by one skilled in the art, a lens is considered to be “soft” if it can be folded back upon itself without breaking.

Any cationic material known to produce a cationic surface of an ophthalmic device can be used herein. It is particularly useful to employ cationic biocompatible materials herein including both soft and rigid materials commonly used for opthalmic lenses, including contact lenses. The present compositions are applicable for use with all types of contact lenses. The cationic ophthalmic lenses can be daily-disposable lenses, extended-wear lenses, planned-replacement lenses, and disposable lenses.

A wide variety of materials can be used herein, and cationic silicone hydrogel materials are particularly preferred. Hydrogels in general are a well-known class of materials that comprise hydrated, cross-linked polymeric systems containing water in an equilibrium state. Silicone hydrogels generally have a water content greater than about 5 weight percent and more commonly between about 10 to about 80 weight percent. Such materials are usually prepared by polymerizing a mixture containing at least one silicone-containing monomer and at least one hydrophilic monomer. Typically, either the silicone-containing monomer or the hydrophilic monomer functions as a crosslinking agent (a crosslinker being defined as a monomer having multiple polymerizable functionalities) or a separate crosslinker may be employed.

Representative examples of applicable cationic silicon-containing monomeric units include cationic monomers of formula I:

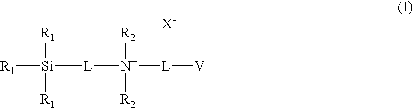

wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof.

X⁻ is at least a single charged counter ion. Examples of single charge counter ions include the group consisting of Cl⁻, Br⁻, I⁻, CF₃CO₂⁻, CH₃CO₂⁻, HCO₃⁻, CH₃SO₄⁻, p-toluenesulfonate, HSO₄⁻, H₂PO₄⁻, NO₃⁻, and CH₃CH(OH)CO₂⁻. Examples of dual charged counter ions would include SO₄²⁻, CO₃²⁻ and HPO₄²⁻. Other charged counter ions would be obvious to one of ordinary skill in the art. It should be understood that a residual amount of counter ion may be present in the hydrated product. Therefore, the use of toxic counterions is to be discouraged. Likewise, it should be understood that, for a singularly charged counterion, the ratio of counterion and quaternary siloxanyl will be 1:1. Counterions of greater negative charge will result in differing ratios based upon the total charge of the counterion.

R₁ and R₂ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group and V is independently a polymerizable ethylenically unsaturated organic radical.

Preferred monomers of formula I are shown in formula II below:

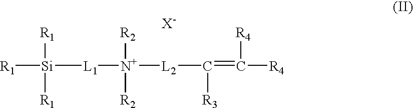

wherein each R₁ is the same and is —OSi(CH₃)₃ R₂ is methyl, L₁ is an alkyl amide, L₂ is a alkyl amide or ester having 2 or 3 carbon atoms that is joined to a polymerizable vinyl group, R₃ is methyl, R₄ is H and X⁻ is Br⁻ or Cl⁻.

Further preferred structures have the following structural formulae III-VII:

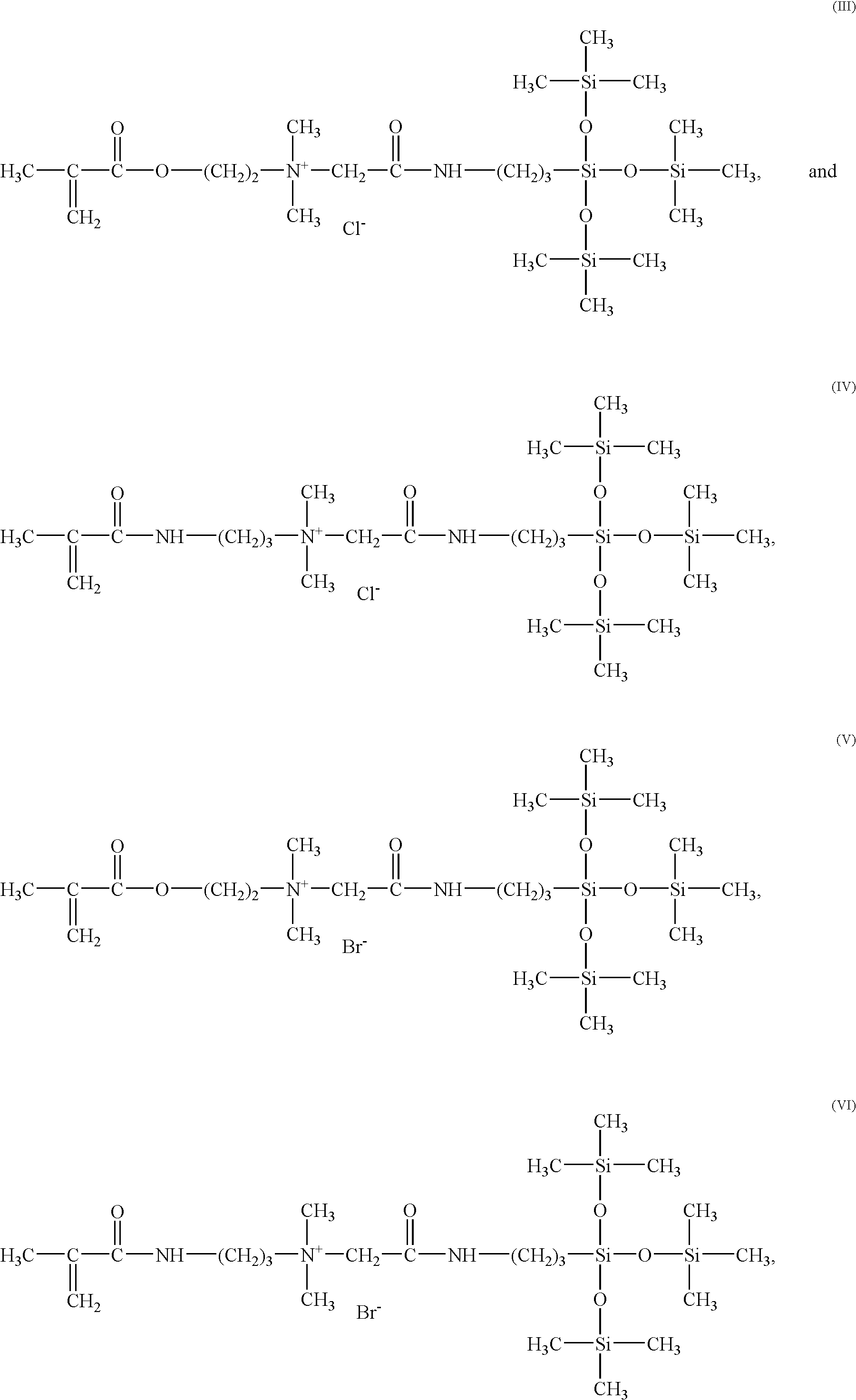

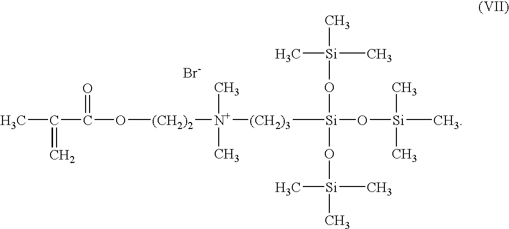

A schematic representation of synthetic methods for making a cationic silicon-containing monomer as disclosed hereinabove is provided below:

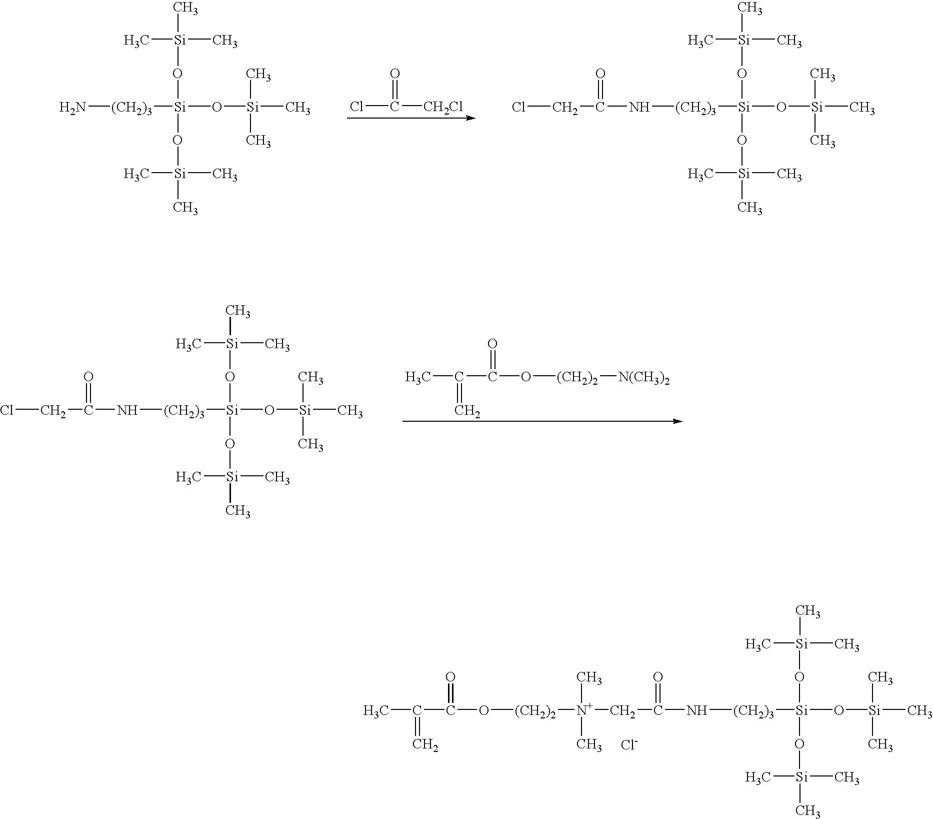

Another class of examples of applicable cationic silicon-containing monomeric units for use herein include cationic monomers of formula VIII:

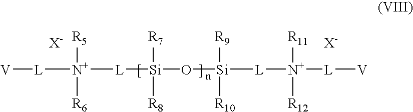

wherein each L can be the same or different and is as defined above for L in formula I; X⁻ is at least a single charged counter ion as defined above for X⁻ in formula I; R₅, R₆, R₇, R₈, R₉, R₁₀, R₁₁ and R₁₂ are each independently as defined above for R₁ in formula I; V is independently a polymerizable ethylenically unsaturated organic radical and n is an integer of 1 to about 300.

Preferred monomers of formula VIII are shown in formulae IX-XIII below:

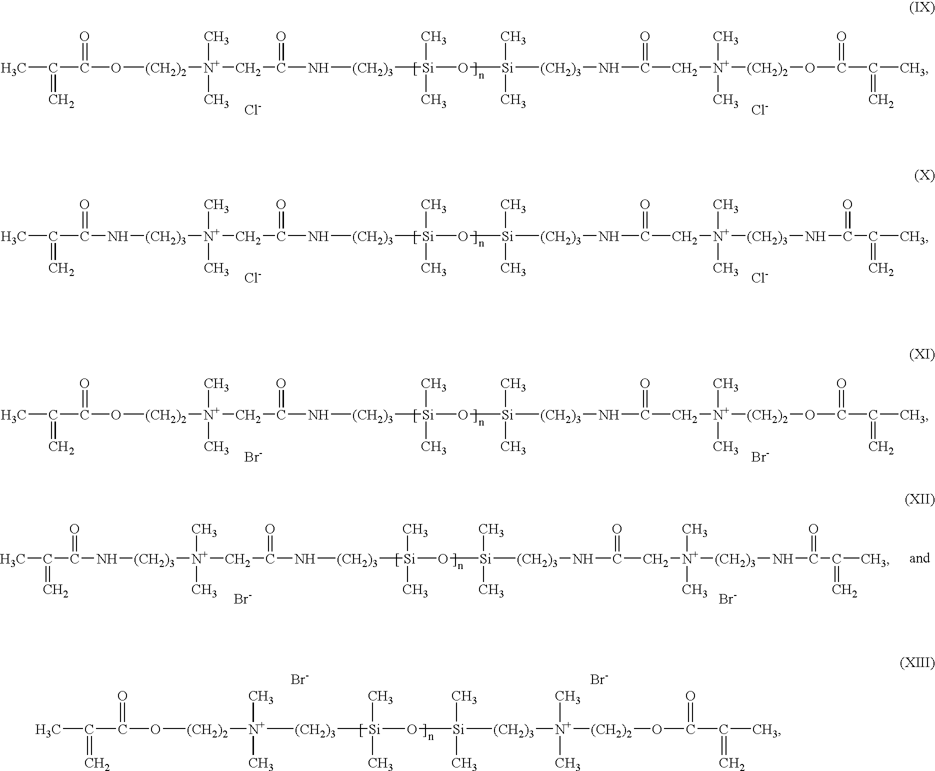

A schematic representation of a synthetic method for making the cationic silicon-containing monomers of formula VIII is provided below:

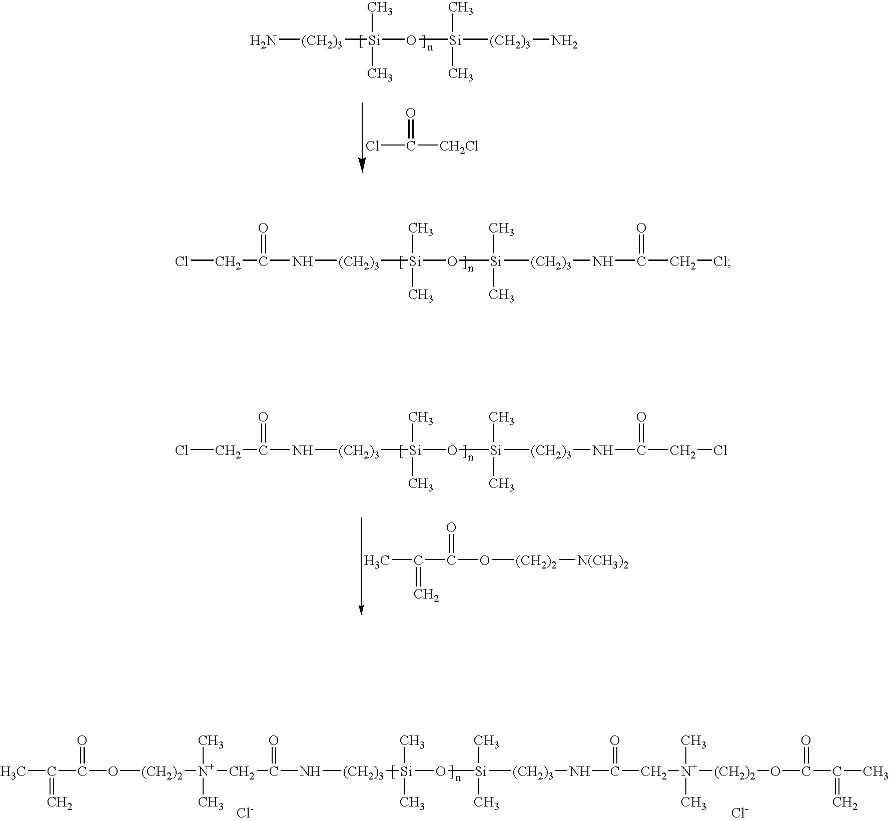

Another class of examples of applicable cationic silicon-containing monomeric units for use herein include cationic monomers of formula XIV:

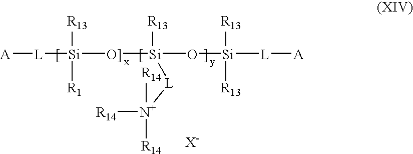

wherein x is 0 to 1000, y is 1 to 300, each L can be the same or different and is as defined above for L in formula I; X⁻ is at least a single charged counter ion as defined above for X⁻ in formula I; each R₁, R₁₃ and R₁₄ are independently as defined above for R₁ in formula I and A is a polymerizable vinyl moiety.

A preferred cationic random copolymer of formula XIV is shown in formula XV below:

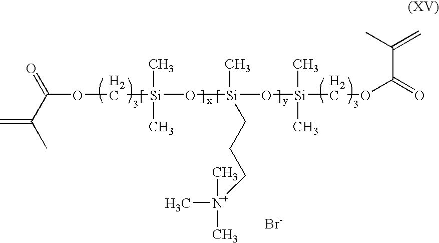

wherein x is 0 to 1000 and y is 1 to 300.

A schematic representation of a synthetic method for making the cationic silicon-containing random copolymers of Formulae XIV and XV is provided below:

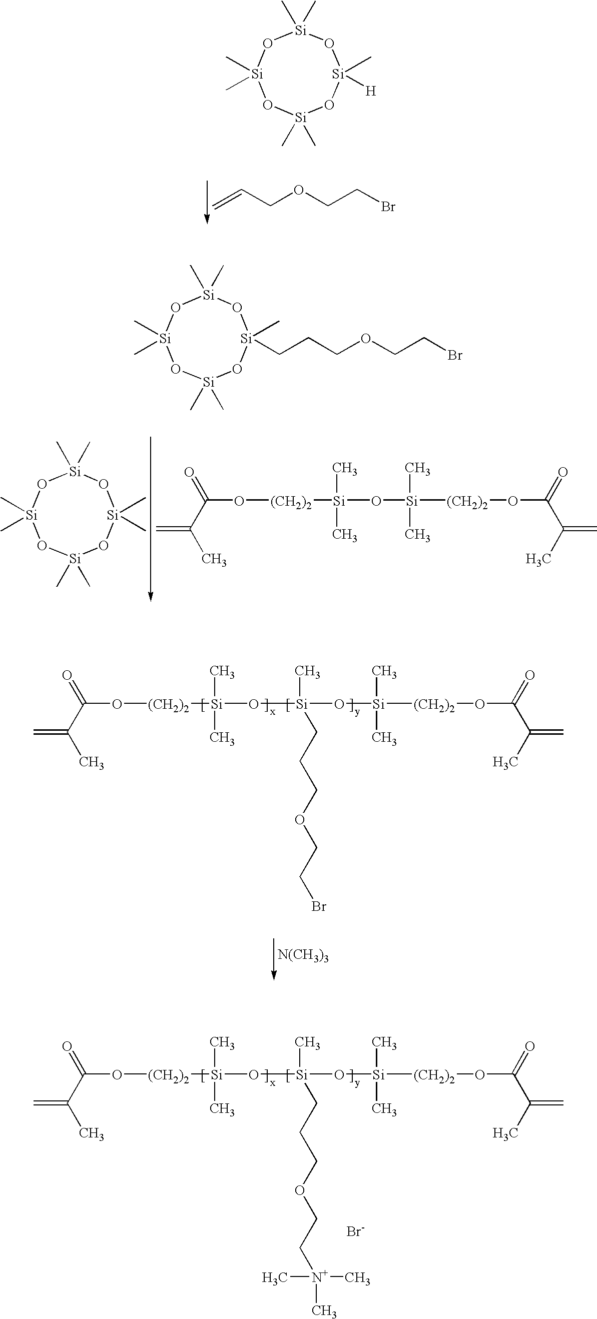

Another class of examples of applicable cationic materials for use herein include cationic random copolymers of formula XVI:

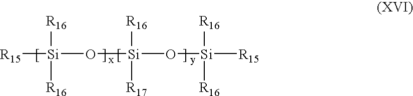

wherein x is 0 to 1000, y is 1 to 300; each R₁₅ and R₁₆ can be the same or different and can be the groups as defined above for R₁ in formula I; R₁₇ is independently one or more of the following formulae XVII and XVIII:

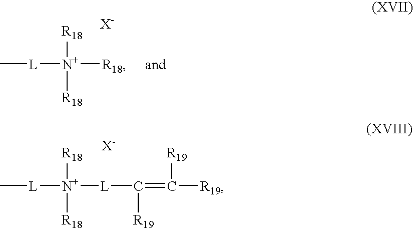

wherein L can be the same or different and is as defined above for L in formula I; X⁻ is at least a single charged counter ion as defined above for X⁻ in formula I; R₁₈ can be the same or different and can be the groups as defined above for R₁ in formula I; and R₁₉ is independently hydrogen or methyl.

A schematic representation of a synthetic method for preparing cationic silicon-containing random copolymers such as poly(dimethylsiloxane) bearing pendant polymerizable cationic groups disclosed herein is provided below.

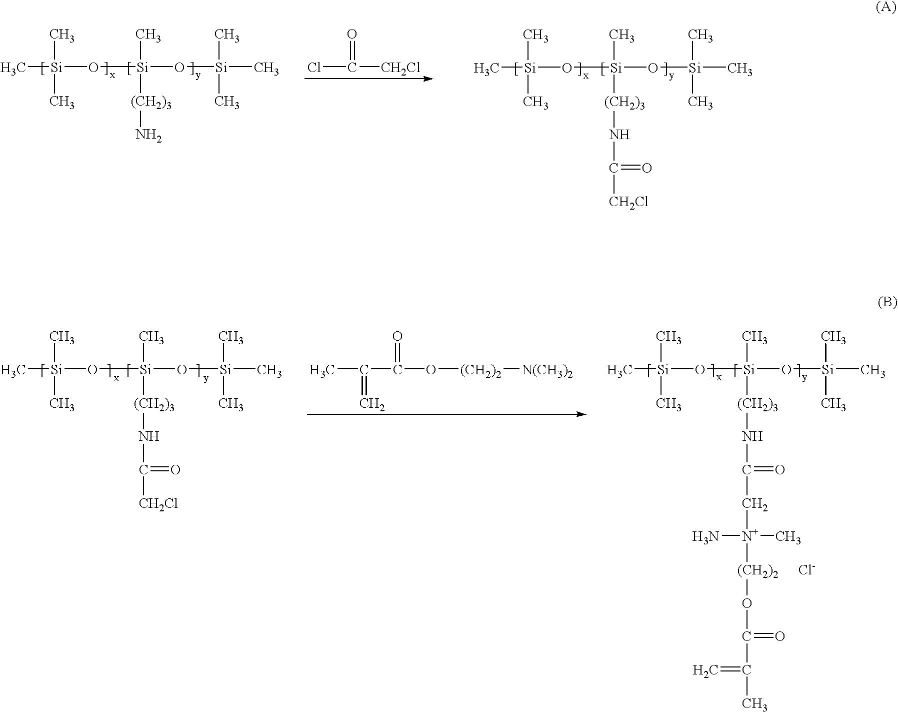

Another synthetic scheme for preparing poly(dimethylsiloxane) bearing pendant cationic groups and pendant polymerizable cationic groups is provided below.

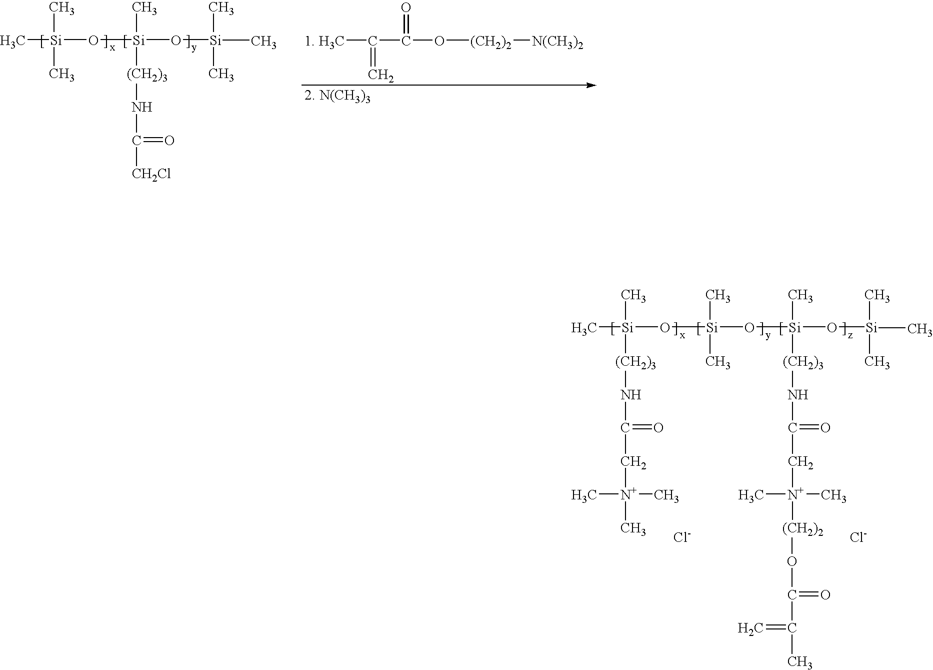

Yet another synthetic scheme for preparing poly(dimethylsiloxane) bearing pendant polymerizable groups and pendant cationic groups is provided below.

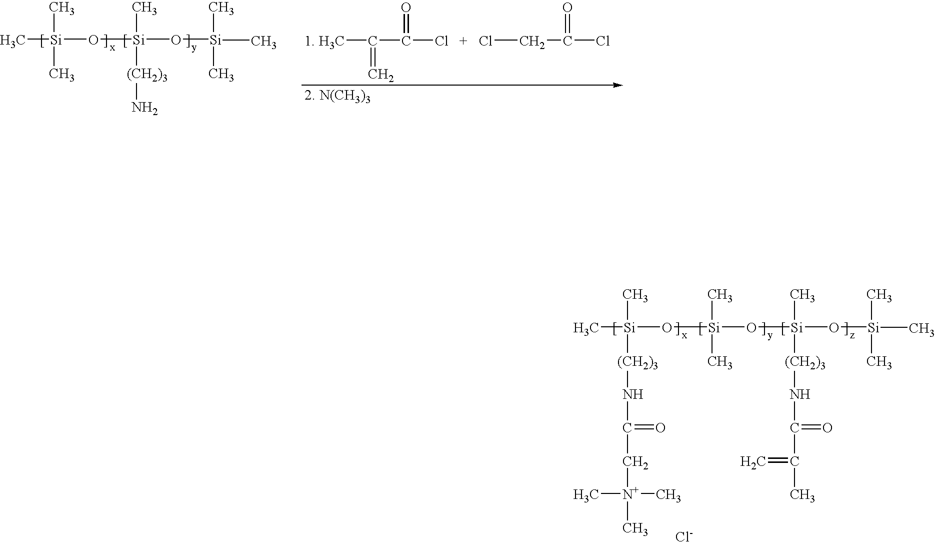

Representative examples of urethanes for use herein include, by way of example, a secondary amine linked to a carboxyl group which may also be linked to a further group such as an alkyl. Likewise the secondary amine may also be linked to a further group such as an alkyl.

Representative examples of carbonates for use herein include, by way of example, alkyl carbonates, aryl carbonates, and the like.

Representative examples of carbamates, for use herein include, by way of example, alkyl carbamates, aryl carbamates, and the like.

Representative examples of carboxyl ureidos, for use herein include, by way of example, alkyl carboxyl ureidos, aryl carboxyl ureidos, and the like.

Representative examples of sulfonyls for use herein include, by way of example, alkyl sulfonyls, aryl sulfonyls, and the like.

Representative examples of alkyl groups for use herein include, by way of example, a straight or branched hydrocarbon chain radical containing carbon and hydrogen atoms of from 1 to about 18 carbon atoms with or without unsaturation, to the rest of the molecule, e.g., methyl, ethyl, n-propyl, 1-methylethyl (isopropyl), n-butyl, n-pentyl, etc., and the like.

Representative examples of fluoroalkyl groups for use herein include, by way of example, a straight or branched alkyl group as defined above having one or more fluorine atoms attached to the carbon atom, e.g., —CF₃, —CF₂CF₃, —CH₂CF₃, —CH₂CF₂H, —CF₂H and the like.

Representative examples of ester groups for use herein include, by way of example, a carboxylic acid ester having one to 20 carbon atoms and the like.

Representative examples of ether or polyether-containing groups for use herein include, by way of example, an alkyl ether, cycloalkyl ether, cycloalkylalkyl ether, cycloalkenyl ether, aryl ether, arylalkyl ether wherein the alkyl, cycloalkyl, cycloalkylalkyl, cycloalkenyl, aryl, and arylalkyl groups are as defined herein, e.g., alkylene oxides, poly(alkylene oxide)s such as ethylene oxide, propylene oxide, butylene oxide, poly(ethylene oxide)s, poly(ethylene glycol)s, poly(propylene oxide)s, poly(butylene oxide)s and mixtures or copolymers thereof, an ether or polyether group of the general formula —R₂₀OR₂₁, wherein R₂₀ is a bond, an alkyl, cycloalkyl or aryl group as defined herein and R₂₁ is an alkyl, cycloalkyl or aryl group as defined herein, e.g., in the case of L as defined in formula I, the ether-containing group can be —CH₂CH₂OC₆H₄— or —CH₂CH₂OC₂H₄—; or in the case of R₁ and R₂ as defined in formula I, the ether-containing group can be —CH₂CH₂OC6H₅ or —CH₂CH₂OC₂H₅, and the like.

Representative examples of amide groups for use herein include, by way of example, an amide of the general formula —R₂₃C(O)NR₂₄R₂₅ wherein R₂₃, R₂₄ and R₂₅ are independently C₁-C₃₀ hydrocarbons, e.g., R₂₃ can be an alkylene group, arylene group, cycloalkylene group and R₂₄ and R₂₅ can be an alkyl groups, aryl group, and cycloalkyl group as defined herein and the like.

Representative examples of amine groups for use herein include, by way of example, an amine of the general formula —R₂₆NR₂₇R₂₈ wherein R₂₆ is a C₂-C₃₀ alkylene, arylene, or cycloalkylene and R₂₇ and R₂8 are independently C₁-C₃₀ hydrocarbons such as, for example, alkyl groups, aryl groups, or cycloalkyl groups as defined herein, and the like.

Representative examples of an ureido group for use herein include, by way of example, an ureido group having one or more substituents or unsubstituted ureido. The ureido group preferably is an ureido group having 1 to 12 carbon atoms. Examples of the substituents include alkyl groups and aryl groups. Examples of the ureido group include 3-methylureido, 3,3-dimethylureido, and 3-phenylureido.

Representative examples of alkoxy groups for use herein include, by way of example, an alkyl group as defined above attached via oxygen linkage to the rest of the molecule, i.e., of the general formula —OR₂₉, wherein R₂₉ is an alkyl, cycloalkyl, cycloalkylalkyl, cycloalkenyl, aryl or an arylalkyl as defined above, e.g., —OCH₃, —OC₂H₅, or —OC₆H₅, and the like.

Representative examples of cycloalkyl groups for use herein include, by way of example, a substituted or unsubstituted non-aromatic mono or multicyclic ring system of about 3 to about 18 carbon atoms such as, for example, cyclopropyl, cyclobutyl, cyclopentyl, cyclohexyl, perhydronapththyl, adamantyl and norbornyl groups bridged cyclic group or spirobicyclic groups, e.g., sprio-(4,4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 18 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 18 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 25 carbon atoms such as, for example, phenyl, naphthyl, tetrahydronapthyl, indenyl, biphenyl and the like, optionally containing one or more heteroatoms, e.g., O and N, and the like.

Representative examples of arylalkyl groups for use herein include, by way of example, a substituted or unsubstituted aryl group as defined above directly bonded to an alkyl group as defined above, e.g., —CH₂C₆H₅, —C₂H₅C₆H₅ and the like, wherein the aryl group can optionally contain one or more heteroatoms, e.g., O and N, and the like.

Representative examples of fluoroaryl groups for use herein include, by way of example, an aryl group as defined above having one or more fluorine atoms attached to the aryl group.

Representative examples of heterocyclic ring groups for use herein include, by way of example, a substituted or unsubstituted stable 3 to about 15 membered ring radical, containing carbon atoms and from one to five heteroatoms, e.g., nitrogen, phosphorus, oxygen, sulfur and mixtures thereof. Suitable heterocyclic ring radicals for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, isochromanyl and the like and mixtures thereof.

Representative examples of heteroaryl groups for use herein include, by way of example, a substituted or unsubstituted heterocyclic ring radical as defined above. The heteroaryl ring radical may be attached to the main structure at any heteroatom or carbon atom that results in the creation of a stable structure.

Representative examples of heteroarylalkyl groups for use herein include, by way of example, a substituted or unsubstituted heteroaryl ring radical as defined above directly bonded to an alkyl group as defined above. The heteroarylalkyl radical may be attached to the main structure at any carbon atom from the alkyl group that results in the creation of a stable structure.

Representative examples of heterocyclo groups for use herein include, by way of example, a substituted or unsubstituted heterocylic ring radical as defined above. The heterocyclo ring radical may be attached to the main structure at any heteroatom or carbon atom that results in the creation of a stable structure.

Representative examples of heterocycloalkyl groups for use herein include, by way of example, a substituted or unsubstituted heterocylic ring radical as defined above directly bonded to an alkyl group as defined above. The heterocycloalkyl radical may be attached to the main structure at carbon atom in the alkyl group that results in the creation of a stable structure.

Representative examples of a “polymerizable ethylenically unsaturated organic radicals” include, by way of example, (meth)acrylate-containing radicals, (meth)acrylamide-containing radicals, vinylcarbonate-containing radicals, vinylcarbamate-containing radicals, styrene-containing radicals and the like. In one embodiment, a polymerizable ethylenically unsaturated organic radical can be represented by the general formula:

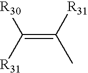

wherein R₃₀ is independently hydrogen, fluorine, an alkyl radical having 1 to 6 carbon atoms, or a —CO—Y—R₃₂ radical wherein Y is —O—, —S— or —NH— and R₃₂ is a divalent alkylene radical having 1 to about 10 carbon atoms; and R₃₁ is hydrogen, fluorine or methyl.

The substituents in the ‘substituted alkyl’, ‘substituted alkoxy’, ‘substituted cycloalkyl’, ‘substituted cycloalkylalkyl’, ‘substituted cycloalkenyl’, ‘substituted arylalkyl’, ‘substituted aryl’, ‘substituted heterocyclic ring’, ‘substituted heteroaryl ring,’ ‘substituted heteroarylalkyl’, ‘substituted heterocycloalkyl ring’, ‘substituted cyclic ring’ and ‘substituted carboxylic acid derivative’ may be the same or different and include one or more substituents such as hydrogen, hydroxy, halogen, carboxyl, cyano, nitro, oxo (=O), thio(=S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocycloalkyl ring, substituted or unsubstituted heteroarylalkyl, substituted or unsubstituted heterocyclic ring, substituted or unsubstituted guanidine, —COORx, —C(O)Rx, —C(S)Rx, —C(O)NRxRy, —C(O)ONRxRy, —NRxCONRyRz, —N(Rx)SORy, —N(Rx)SO2Ry, —(═N—N(Rx)Ry), —NRxC(O)ORy, —NRxRy, —NRxC(O)Ry-, —NRxC(S)Ry —NRxC(S)NRyRz, —SONRxRy-, —SO2NRxRy-, —ORx, —ORxC(O)NRyRz, —ORxC(O)ORy-, —OC(O)Rx, —OC(O)NRxRy, -RxNRyC(O)Rz, -RxORy, -RxC(O)ORy, -RxC(O)NRyRz, -RxC(O)Rx, -RxOC(O)Ry, —SRx, —SORx, —SO2Rx, —ONO2, wherein Rx, Ry and Rz in each of the above groups can be the same or different and can be a hydrogen atom, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, ‘substituted heterocycloalkyl ring’ substituted or unsubstituted heteroarylalkyl, or a substituted or unsubstituted heterocyclic ring.

The above silicone materials are merely exemplary, and other materials for use as cationic substrates that can benefit by being stored in a solution containing an anionic polymer according to the present invention and have been disclosed in various publications and are being continuously developed for use in contact lenses and other medical devices can also be used. For example, other suitable cationic monomer materials have a molecular weight of about 600 grams per mole or less, and include a quaternary ammonium group or a tertiary amine group that can be protonated at a pH value of about 7.2 to about 7.4 (physiological pH values). Illustrative monomers include the tertiary C₁-C₁₀ alkyl, C₂-C₃ alkanol, and benzyl aminoethyl or N-morpholinoethyl esters of acrylates and methacrylates such as 2-dimethylaminoethyl methacrylate (DMEAM), 2-N-morpholinoethyl methacrylate (MEM), N,N-diethanolaminoethyl methacrylate, N,N-dimethoxyethylaminoethyl methacrylate, vinyl amine, aminostyrene, 2-vinyl pyridine, 4-vinyl pyridine, N-(2-vinyloxyethyl)piperidine and quaternary ammonium compounds such as 3-trimethylammonium-2-hydroxypropyl methacrylate chloride (TMAHPM), 2-trimethylammoniumethyl methacrylic hydroxide, 2-trimethylammoniumethyl acrylic hydroxide, 2-trimethyl-ammoniummethyl methacrylic chloride, 2-trimethylammoniummethyl acrylic chloride, and 2-methacryloyloxy ethyltrimethylammonium methyl sulfate.

The cationic ophthalmic lens having a cationic surface to be packaged herein can be formed from a cationic ophthalmic lens-forming monomeric mixture containing at least one or more cationic materials such as those described hereinabove. According to preferred embodiments, the cationic ophthalmic lens is a polymerization product of a mixture comprising one or more of the aforementioned cationic materials and at least a second monomer.

Useful cationic ophthalmic lens made with the cationic materials may require hydrophobic, possibly other silicon containing monomers. Preferred compositions have both hydrophilic and hydrophobic monomers. Especially preferred is silicon-containing hydrogels.

Silicon-containing hydrogels are prepared by polymerizing a mixture containing at least one silicon-containing monomer and at least one hydrophilic monomer. The silicon-containing monomer may function as a crosslinking agent (a crosslinker being defined as a monomer having multiple polymerizable functionalities) or a separate crosslinker may be employed.

An early example of a silicon-containing contact lens material is disclosed in U.S. Pat. No. 4,153,641. Lenses are made from poly(organosiloxane) monomers which are α, ω terminally bonded through a divalent hydrocarbon group to a polymerized activated unsaturated group. Various hydrophobic silicon-containing prepolymers such as 1,3-bis(methacryloxyalkyl)polysiloxanes were copolymerized with known hydrophilic monomers such as 2-hydroxyethyl methacrylate (HEMA).

U.S. Pat. No. 5,358,995 discloses a silicon containing hydrogel which is comprised of an acrylic ester-capped polysiloxane prepolymer, polymerized with a bulky polysiloxanylalkyl(meth)acrylate monomer, and at least one hydrophilic monomer. The acrylic ester-capped polysiloxane prepolymer, commonly known as M₂Dₓ consists of two acrylic ester end groups and “x” number of repeating dimethylsiloxane units. The preferred bulky polysiloxanylalkyl(meth)acrylate monomers are TRIS-type (methacryloxypropyltris(trimethylsiloxy)silane) with the hydrophilic monomers being either acrylic- or vinyl-containing.

Other examples of silicon-containing monomer mixtures which may be used with this invention include the following: vinyl carbonate and vinyl carbamate monomer mixtures as disclosed in U.S. Pat. Nos. 5,070,215 and 5,610,252; fluorosilicon monomer mixtures as disclosed in U.S. Pat. Nos. 5,321,108; 5,387,662 and 5,539,016; fumarate monomer mixtures as disclosed in U.S. Pat. Nos. 5,374,662; 5,420,324 and 5,496,871 and urethane monomer mixtures as disclosed in U.S. Pat. Nos. 5,451,651; 5,639,908; 5,648,515 and 5,594,085, all of which are commonly assigned to assignee herein Bausch & Lomb Incorporated, and the entire disclosures of which are incorporated herein by reference.

Examples of non-silicon hydrophobic materials include alkyl acrylates and methacrylates.

The cationic materials such as cationic silicon-containing monomers may be copolymerized with a wide variety of hydrophilic monomers to produce silicon hydrogel lenses. Suitable hydrophilic monomers include: unsaturated carboxylic acids, such as methacrylic and acrylic acids; acrylic substituted alcohols, such as 2-hydroxyethyl methacrylate and 2-hydroxyethyl acrylate; vinyl lactams, such as N-vinylpyrrolidone (NVP) and 1-vinylazonan-2-one; and acrylamides, such as methacrylamide and N,N-dimethylacrylamide (DMA).

Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art.

Hydrophobic crosslinkers would include methacrylates such as ethylene glycol dimethacrylate (EGDMA) and allyl methacrylate (AMA).

If desired, an organic diluent may be included in the initial monomeric mixture. As used herein, the term “organic diluent” encompasses organic compounds which minimize incompatibility of the components in the initial monomeric mixture and are substantially nonreactive with the components in the initial mixture. Additionally, the organic diluent serves to minimize phase separation of polymerized products produced by polymerization of the monomeric mixture.

Contemplated organic diluents include tert-butanol (TBA); diols such as ethylene glycol, and polyols such as glycerol. Preferably, the organic diluent is sufficiently soluble in the extraction solvent to facilitate its removal from a cured article during the extraction step. Other suitable organic diluents would be apparent to a person of ordinary skill in the art.

The organic diluent is included in an amount effective to provide the desired effect. Generally, the diluent is included at about 5 to about 60% by weight of the monomeric mixture, with about 10 to about 50% by weight being especially preferred.

Lens formation can be by free radical polymerization such as azobisisobutyronitrile (AIBN) and peroxide catalysts using initiators and under conditions such as those set forth in U.S. Pat. No. 3,808,179, the contents of which are incorporated by reference herein. Photoinitiation of polymerization of the monomer mixture as is well known in the art may also be used in the process of forming an article as disclosed herein. Colorants and the like may be added prior to monomer polymerization.

Contact lenses for application of the present invention can be manufactured employing various conventional techniques, to yield a shaped article having the desired posterior and anterior lens surfaces. Spincasting methods are disclosed in U.S. Pat. Nos. 3,408,429 and 3,660,545; and static casting methods are disclosed in U.S. Pat. Nos. 4,113,224, 4,197,266 and 5,271,876. Curing of the monomeric mixture may be followed by a machining operation in order to provide a contact lens having a desired final configuration. As an example, U.S. Pat. No. 4,555,732 discloses a process in which an excess of a monomeric mixture is cured by spincasting in a mold to form a shaped article having an anterior lens surface and a relatively large thickness. The posterior surface of the cured spincast article is subsequently lathe cut to provide a contact lens having the desired thickness and posterior lens surface. Further machining operations may follow the lathe cutting of the lens surface, for example, edge-finishing operations.

Typically, an organic diluent is included in the initial monomeric mixture in order to minimize phase separation of polymerized products produced by polymerization of the monomeric mixture and to lower the glass transition temperature of the reacting polymeric mixture, which allows for a more efficient curing process and ultimately results in a more uniformly polymerized product. Sufficient uniformity of the initial monomeric mixture and the polymerized product is of particular importance for silicone hydrogels, primarily due to the inclusion of silicone-containing monomers which may tend to separate from the hydrophilic comonomer. Suitable organic diluents include, for example, monohydric alcohols such as C₆-C₁₀ straight-chained aliphatic monohydric alcohols, e.g., n-hexanol and n-nonanol; diols such as ethylene glycol; polyols such as glycerin; ethers such as diethylene glycol monoethyl ether; ketones such as methyl ethyl ketone; esters such as methyl enanthate; and hydrocarbons such as toluene. Preferably, the organic diluent is sufficiently volatile to facilitate its removal from a cured article by evaporation at or near ambient pressure. Generally, the diluent may be included at about 5 to about 60 percent by weight of the monomeric mixture, with about 10 to about 50 percent by weight being preferred. If necessary, the cured lens may be subjected to solvent removal, which can be accomplished by evaporation at or near ambient pressure or under vacuum. An elevated temperature can be employed to shorten the time necessary to evaporate the diluent.

Following removal of the organic diluent, the lens can then be subjected to mold release and optional machining operations. The machining step includes, for example, buffing or polishing a lens edge and/or surface. Generally, such machining processes may be performed before or after the article is released from a mold part. As an example, the lens may be dry released from the mold.

Next, the device will be immersed in a packaging solution and stored in a packaging system according to the present invention. Generally, a packaging system for the storage of a cationic ophthalmic device such as a lens according to the present invention includes at least a sealed container containing one or more unused cationic ophthalmic lenses immersed in an aqueous packaging solution. Preferably, the sealed container is a hermetically sealed blister-pack, in which a concave well containing a contact lens is covered by a metal or plastic sheet adapted for peeling in order to open the blister-pack. The sealed container may be any suitable generally inert packaging material providing a reasonable degree of protection to the lens, preferably a plastic material such as polyalkylene, PVC, polyamide, and the like.

The aqueous packaging solution will contain at least an effective amount of one or more anionic polymers. Due to the relatively strong electrostatic interaction between cationic and anionic groups, it is believed that the cationic ophthalmic lens can be treated with the solution to create an electrostatic binding between the anionic polymer and the surface of the lens to modify the surface properties of the lens resulting in a lens having improved wettability, lubricity and microbial properties. If desired, the anionic polymer can potentially be refreshed on the surface of the lens during use in the eye by repeated treatments, if necessary.

Any suitable anionic polymer may be employed in accordance with the present invention provided that it is capable of modifying the surface properties of the cationic ophthalmic lens resulting in improved wettability, lubricity and microbial properties and has no substantial detrimental effect on the lens being stored or on the wearer of the lens. The anionic polymer is preferably ophthalmically acceptable at the concentrations used. The anionic polymer can include two (2) or more anionic (or negative) charges and preferably three (3) or more anionic (or negative) charges. It is preferred that each of the repeating units of the polymeric material include a discrete anionic charge. Particularly useful anionic polymers are those which are water soluble, for example, soluble at the concentrations used in the presently useful liquid aqueous media, such as a liquid aqueous medium containing the anionic polymer. Particularly useful anionic polymers are those which are not eliminated during terminal sterilization of the packaged lenses.

In one embodiment, a class of anionic polymers includes one or more polymeric materials having multiple anionic charges. Representative examples of suitable anionic polymers for use herein include, but are not limited to, hyaluronic acid or a derivative thereof and/or salts thereof; metal carboxymethylcelluloses; metal carboxymethylhydroxyethylcelluloses; metal carboxymethylstarchs; metal carboxymethylhydroxyethyl starches; hydrolyzed polyacrylamides; polyacrylonitriles; heparin; homopolymers and copolymers of one or more of acrylic and methacrylic acids, metal acrylates and methacrylates; alginic acid; metal alginates; vinylsulfonic acid; metal vinylsulfonate; amino acids such as aspartic acid, glutamic acid and the like; metal salts of amino acids; p-styrenesulfonic acid; metal p-styrenesulfonate; 2-methacryloyloxyethylsulfonic acids; metal 2-methacryloyloxethylsulfonates; 3-methacryloyloxy-2-hydroxypropylsulfonic acids; metal 3-methacryloyloxy-2-hydroxypropylsulfonates; 2-acrylamido-2-methylpropanesulfonic acids; metal 2-acrylamido-2-methylpropanesulfonates; allylsulfonic acid; metal allylsulfonate and the like. In one embodiment, an anionic polymer is an anionic polysaccharide. In another embodiment, an anionic polymer includes one or more of polyacrylic acid, polymethacrylic acid, polyethylene amine, polysaccharides, alginic acid, pectinic acid, carboxy methyl cellulose, hyaluronic acid, heparin, chitosan, carboxymethyl chitosan, carboxymethyl starch, carboxymethyl dextran, heparin sulfate, chondroitin sulfate, cationic guar, any salts thereof, and mixtures thereof. The above list is intended for illustrative purposes only and not to limit the scope of the present invention. Such polymers are known to those of skill in the art.

In another embodiment, an anionic polymer includes one or more cellulose derivative, anionic polymers derived from acrylic acid (e.g., polymers derived from acrylic acid, acrylates and the like and mixtures thereof), anionic polymers derived from methacrylic acid (e.g., polymers derived from methacrylic acid, methacrylates, and the like and mixtures thereof), anionic polymers derived from alginic acid (e.g., polymers derived from alginic acid, alginates, and the like and mixtures thereof), anionic polymers derived from amino acids (e.g., polymers derived from amino acids, amino acid salts, and the like and mixtures thereof) and mixtures thereof. Particularly useful anionic polymers for use herein include cellulose polymers such as carboxymethylcelluloses.

The amount of anionic polymer employed is that amount effective to improve the surface properties of the cationic lens being stored therein. Preferably the anionic polymer is present in the aqueous packaging solution of the invention in an amount of at least 0.01% w/v. The specific amount of such anionic polymers used can vary widely depending on a number of factors, for example, the specific anionic polymer being employed. In addition, excessive amounts of anionic polymer are preferably to be avoided since this may be wasteful and unnecessary and may have an adverse impact on the wearer of the disinfected contact lens. Preferably, the anionic polymer is present in the aqueous packaging solution in an amount of at least about 0.01% w/v, preferably about 0.05% w/v to about 5% w/v or about 0.1% w/v to about 1% w/v.

The aqueous packaging solutions according to the present invention are physiologically compatible. Specifically, the solution must be “ophthalmically safe” for use with a lens such as a cationic contact lens, meaning that a contact lens treated with the solution is generally suitable and safe for direct placement on the eye without rinsing, that is, the solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations. The solution should be sterile in that the absence of microbial contaminants in the product prior to release must be statistically demonstrated to the degree necessary for such products. The liquid media useful in the present invention are selected to have no substantial detrimental effect on the lens being treated or cared for and to allow or even facilitate the present lens treatment or treatments. The liquid media are preferably aqueous-based. A particularly useful aqueous liquid medium is that derived from saline, for example, a conventional saline solution or a conventional buffered saline solution.

The pH of the present aqueous packaging solutions should be maintained within the range of about 6.0 to about 9, and preferably about 6.5 to about 7.8. Suitable buffers may be added, such as boric acid, sodium borate, potassium citrate, citric acid, sodium bicarbonate, tromethamine, and various mixed phosphate buffers (including combinations of Na₂ HPO₄, NaH₂ PO₄ and KH₂ PO₄) and mixtures thereof. Generally, buffers will be used in amounts ranging from about 0.05 to about 2.5 percent by weight, and preferably from about 0.1 to about 1.5 percent by weight of the solution.

Typically, the solutions for use on the packages of the present invention are also adjusted with tonicity agents, to approximate the osmotic pressure of normal lacrimal fluids which is equivalent to a 0.9 percent solution of sodium chloride or 2.5 percent of glycerol solution. The solutions are made substantially isotonic with physiological saline used alone or in combination, otherwise if simply blended with sterile water and made hypotonic or made hypertonic the lenses may lose their desirable optical parameters. Correspondingly, excess saline may result in the formation of a hypertonic solution which will cause stinging and eye irritation.

Examples of suitable tonicity adjusting agents include, but are not limited to, sodium and potassium chloride, dextrose, glycerin, calcium and magnesium chloride and the like and mixtures thereof. These agents are typically used individually in amounts ranging from about 0.01 to about 2.5% w/v and preferably from about 0.2 to about 1.5% w/v. Preferably, the tonicity agent will be employed in an amount to provide a final osmotic value of at least about 200 mOsm/kg, preferably from about 200 to about 400 mOsm/kg, more preferably from about 250 to about 350 mOsm/kg, and most preferably from about 280 to about 320 mOsm/kg.

If desired, one or more additional components can be included in the packaging solution. Such additional component or components are chosen to impart or provide at least one beneficial or desired property to the packaging solution. Such additional components may be selected from components which are conventionally used in one or more contact lens care compositions. Examples of such additional components include cleaning agents, wetting agents, nutrient agents, sequestering agents, viscosity builders, contact lens conditioning agents, antioxidants, and the like and mixtures thereof. These additional components may each be included in the packaging solutions in an amount effective to impart or provide the beneficial or desired property to the packaging solutions. For example, such additional components may be included in the packaging solutions in amounts similar to the amounts of such components used in other, e.g., conventional, contact lens care products.

Useful sequestering agents include, but are not limited to, disodium ethylene diamine tetraacetate, alkali metal hexametaphosphate, citric acid, sodium citrate and the like and mixtures thereof.

Useful viscosity builders include, but are not limited to, hydroxyethyl cellulose, hydroxymethyl cellulose, polyvinyl pyrrolidone, polyvinyl alcohol and the like and mixtures thereof.

Useful antioxidants include, but are not limited to, sodium metabisulfite, sodium thiosulfate, N-acetylcysteine, butylated hydroxyanisole, butylated hydroxytoluene and the like and mixtures thereof.

The method of packaging and storing a cationic ophthalmic lens such as a cationic contact lens according to the present invention includes at least packaging a cationic ophthalmic lens immersed in the aqueous packaging solution described above. The method may include immersing the cationic ophthalmic lens in an aqueous solution prior to delivery to the customer/wearer, directly following manufacture of the contact lens. Alternately, the packaging and storing in the solution of the present invention may occur at an intermediate point before delivery to the ultimate customer (wearer) but following manufacture and transportation of the lens in a dry state, wherein the dry lens is hydrated by immersing the lens in the contact-lens packaging solution. Consequently, a package for delivery to a customer may include a sealed container containing one or more unused lenses immersed in an aqueous packaging solution according to the present invention.

In one embodiment, the steps leading to the present packaging system includes (1) molding a cationic ophthalmic lens in a mold comprising a posterior and anterior mold portion, (2) removing the lens from the mold and hydrating the lens, (3) introducing the packaging solution with one or more of the anionic polymers into the container with the lens supported therein, and (4) sealing the container. Preferably, the method also includes the step of sterilizing the contents of the container. Sterilization may take place prior to, or most conveniently after, sealing of the container and may be effected by any suitable method known in the art, e.g., by autoclaving of the sealed container and its contents at temperatures of about 120° C. or higher.

The following non-limiting examples illustrate certain aspects of the present invention.

### EXAMPLES

All solvents and reagents were obtained from Sigma-Aldrich (Milwaukee, Wis.), and used as received with the exception of aminopropyl-terminated poly(dimethylsiloxane), obtained from Gelest, Inc. (Morrisville, Pa.), and 3-methacryloxypropyl tris(trimethylsiloxy)silane, obtained from Silar Laboratories (Scotia, N.Y.), both used without further purification, and the monomers 2-hydroxyethyl methacrylate and 1-vinyl-2-pyrrolidone were purified using standard techniques.

Analytical Measurements

ESI-TOF MS: The electrospray (ESI) time of flight (TOF) MS analysis was performed on an Applied Biosystems Mariner instrument. The instrument operated in positive ion mode. The instrument was mass calibrated with a standard solution containing lysine, angiotensinogen, bradykinin (fragment 1-5) and des-Pro bradykinin. This mixture provides a seven-point calibration from 147 to 921 m/z. The applied voltage parameters were optimized from signal obtained from the same standard solution. For exact mass measurements poly(ethylene glycol) (PEG), having a nominal Mₙ value of 400 Da, was added to the sample of interest and used as an internal mass standard. Two PEG oligomers that bracketed the sample mass of interest were used to calibrate the mass scale. Samples were prepared as 30 μM solutions in isopropanol (IPA) with the addition of 2% by volume saturated NaCl in IPA.

Samples were directly infused into the ESI-TOF MS instrument at a rate of 35 μL/min. A sufficient resolving power (6000 RP m/Δm FWHM) was achieved in the analysis to obtain the monoistopic mass for each sample. In each analysis the experimental monoisotopic mass was compared to the theoretical monoisotopic mass as determined from the respective elemental compositions. In each analysis the monoisotopic mass comparison was less than 10 ppm error. It should be noted that uncharged samples have a sodium (Na) atom included in their elemental composition. This Na atom occurs as a necessary charge agent added in the sample preparation procedure. Some samples do not require an added charge agent since they contain a charge from the quaternary nitrogen inherent to their respective structure.

GC: Gas chromatography was performed using a Hewlett Packard HP 6890 Series GC System. Purities were determined by integration of the primary peak and comparison to the normalized chromatograph.

NMR: ¹H-NMR characterization was carried out using a 400 MHz Varian spectrometer using standard techniques in the art. Samples were dissolved in chloroform-d (99.8 atom % D), unless otherwise noted. Chemical shifts were determined by assigning the residual chloroform peak at 7.25 ppm. Peak areas and proton ratios were determined by integration of baseline separated peaks. Splitting patterns (s=singlet, d=doublet, t=triplet, q=quartet, m=multiplet, br=broad) and coupling constants (J/Hz) are reported when present and clearly distinguishable.

SEC: Size Exclusion Chromatography (SEC) analyses were carried out by injection of 100 μL of sample dissolved in tetrahydrofuran (THR) (5-20 mg/mL) onto a Polymer Labs PL Gel Mixed Bed E (×2) column at 35° C. using a Waters 515 HPLC pump and HPLC grade THF mobile phase flow rate of 1.0 mL/min, and detected by a Waters 410 Differential Refractometer at 35° C. Values of Mₙ, M_{w}, and polydispersity (PD) were determined by comparison to Polymer Lab Polystyrene narrow standards.

Mechanical properties and Oxygen Permeability: Modulus and elongation tests were conducted according to ASTM D-1708a, employing an Instron (Model 4502) instrument where the hydrogel film sample is immersed in borate buffered saline; an appropriate size of the film sample is gauge length 22 mm and width 4.75 mm, where the sample further has ends forming a dog bone shape to accommodate gripping of the sample with clamps of the Instron instrument, and a thickness of 200+50 microns.

Oxygen permeability (also referred to as Dk) was determined by the following procedure. Other methods and/or instruments may be used as long as the oxygen permeability values obtained therefrom are equivalent to the described method. The oxygen permeability of silicone hydrogels is measured by the polarographic method (ANSI Z80.20-1998) using an O2 Permeometer Model 201T instrument (Createch, Albany, Calif. USA) having a probe containing a central, circular gold cathode at its end and a silver anode insulated from the cathode. Measurements are taken only on pre-inspected pinhole-free, flat silicone hydrogel film samples of three different center thicknesses ranging from 150 to 600 microns. Center thickness measurements of the film samples may be measured using a Rehder ET-1 electronic thickness gauge.

Generally, the film samples have the shape of a circular disk. Measurements are taken with the film sample and probe immersed in a bath containing circulating phosphate buffered saline (PBS) equilibrated at 35° C.±0.2°. Prior to immersing the probe and film sample in the PBS bath, the film sample is placed and centered on the cathode premoistened with the equilibrated PBS, ensuring no air bubbles or excess PBS exists between the cathode and the film sample, and the film sample is then secured to the probe with a mounting cap, with the cathode portion of the probe contacting only the film sample. For silicone hydrogel films, it is frequently useful to employ a Teflon polymer membrane, e.g., having a circular disk shape, between the probe cathode and the film sample. In such cases, the Teflon membrane is first placed on the pre-moistened cathode, and then the film sample is placed on the Teflon membrane, ensuring no air bubbles or excess PBS exists beneath the Teflon membrane or film sample. Once measurements are collected, only data with correlation coefficient value (R2) of 0.97 or higher should be entered into the calculation of Dk value.

At least two Dk measurements per thickness, and meeting R2 value, are obtained. Using known regression analyses, oxygen permeability (Dk) is calculated from the film samples having at least three different thicknesses. Any film samples hydrated with solutions other than PBS are first soaked in purified water and allowed to equilibrate for at least 24 hours, and then soaked in PHB and allowed to equilibrate for at least 12 hours. The instruments are regularly cleaned and regularly calibrated using RGP standards. Upper and lower limits are established by calculating a ±8.8% of the Repository values established by William J. Benjamin, et al., The Oxygen Permeability of Reference Materials, Optom Vis Sci 7 (12s): 95 (1997), the disclosure of which is incorporated herein in its entirety:

| | Repository | | |
|---|---|---|---|
| Material Name | Values | Lower Limit | Upper Limit |
| Fluoroperm 30 | 26.2 | 24 | 29 |
| Menicon EX | 62.4 | 56 | 66 |
| Quantum II | 92.9 | 85 | 101 |

### ABBREVIATIONS

NVP 1-Vinyl-2-pyrrolidone

TRIS 3-Methacryloxypropyltris(trimethylsiloxy)silane

HEMA 2-Hydroxyethyl methacrylate

v-64 2,2′-Azobis(2-methylpropionitrile)

EGDMA ethylene glycol dimethacrylate

SA 2-[3-(2H-Benzotriazol-2-yl)-4-hydroxyphenyl]ethyl methacrylate

IMVT 1,4-bis[4-(2-methacryloxyethyl)phenylamino]anthraquinone

Unless otherwise specifically stated or made clear by its usage, all numbers used in the examples should be considered to be modified by the term “about” and to be weight percent.

### Example 1

### Preparation of 3-(chloroacetylamido)propyltris(trimethylsiloxysilane)

To a vigorously stirred, biphasic solution of 3-aminopropyltris(trimethylsiloxy)silane (50 g, 141 mmol) obtained from Gelest, Inc., Morrisville, Pa., in dichloromethane (200 mL) and NaOH_{(aq)} (0.75 M, 245 mL) was added a solution of chloroacetyl chloride (14.6 mL, 0.18 mol) in dichloromethane (80 mL) dropwise at ambient temperature. After 1 additional hour at ambient temperature, the organic layer was separated and stirred 3 h over silica gel (15 g) and an additional half hour over sodium sulfate (15 g). Solvents were removed at reduced pressure to afford the product as a colorless liquid (42 g, 84%): ¹H NMR (CDCl₃, 400 MHz) δ 6.64 (br, 1 H), 4.04 (s, 2 H), 3.30-3.24 (m, 2 H), 1.59-1.51 (m, 2 H), 0.45-0.42 (m, 2 H), 0.08 (s, 27 H); GC: 99.3% purity; ESI-TOF MS data is summarized in Table 1, and the mass spectrum also illustrated the characteristic chlorine isotopic distribution pattern as predicted by the elemental composition.

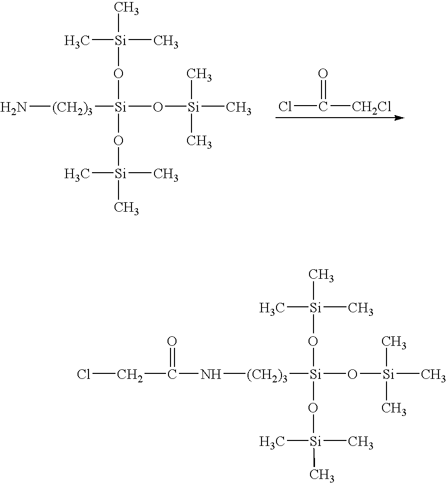

### Example 2

### Preparation of 3-(bromoacetylamido)propyltris(trimethylsiloxysilane)

Bromoacetyl chloride was reacted with 3-aminopropyltris(trimethylsiloxy)silane in substantially the same manner as described in Example 1 to afford the product as a colorless liquid (44.4 g, 79%): ¹H NMR (CDCl₃, 400 MHz) δ 6.55 (br, 1 H), 3.88 (s, 2 H), 3.26 (q, J=7 Hz, 2 H), 1.59-1.51 (m, 2 H), 0.045 (m, 2 H), 0.09 (s, 27 H); GC: 93.2% purity; ESI-TOF MS data is summarized in Table 1, and the mass spectrum also illustrated the characteristic bromine isotopic distribution pattern as predicted by the elemental composition.

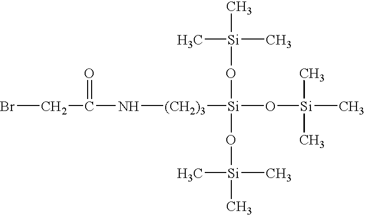

### Example 3

### Preparation of cationic methacrylate chloride Functionalized tris(trimethylsiloxy)silane

To a solution of 3-(chloroacetylamido)propyltris(trimethylsiloxysilane) (10.0 g, 23.2 mmol) from Example 1 above in ethyl acetate (35 mL) was added 2-(dimethylamino)ethyl methacrylate (4.13 mL, 24.5 mmol) and the solution was heated at 60° C. under nitrogen atmosphere with stirring in the dark. Aliquots were removed periodically and monitored for conversion of reagent by ¹H NMR integration. After 35 h the solution was cooled and stripped at reduced pressure to afford cationic methacrylate chloride functionalized tris(trimethylsiloxy)silane (13.8 g, 100%) as a highly viscous liquid: ¹H NMR (CDCl₃, 400 MHz) δ 9.24 (br, 1 H), 6.12 (s, 1 H), 5.66 (s, 1 H), 4.76 (s, 2 H), 4.66-4.64 (m, 2 H), 4.16-4.14 (m, 2 H), 3.46 (s, 6 H), 3.20 (q, J=7 Hz, 2 H), 1.93 (s, 3 H), 1.60-1.52 (m, 2 H), 0.45-0.41 (m, 2 H), 0.07 (s, 27 H); ESI-TOF MS data is summarized in Table 1.

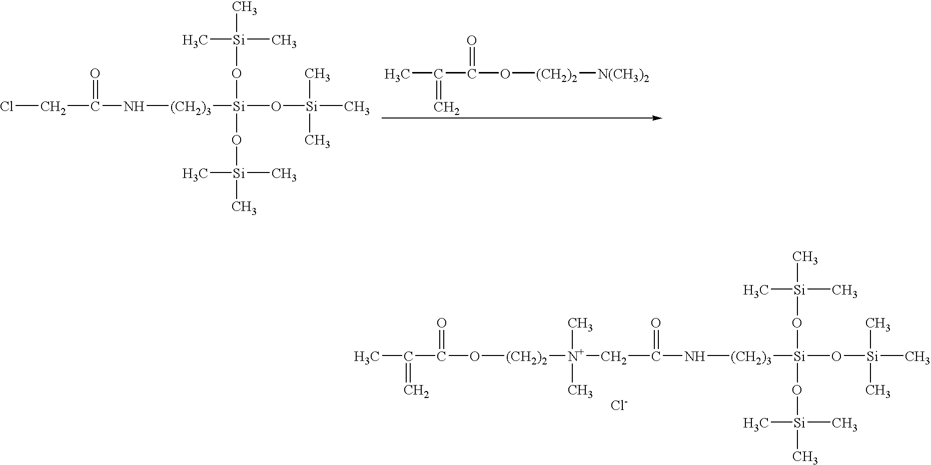

### Example 4

### Preparation of cationic methacrylamide chloride Functionalized tris(trimethylsiloxy)silane

3-(chloroacetylamido)propyltris(trimethylsiloxysilane) (10.0 g, 23.2 mmol) from Example 1 above was reacted with N-[3-(dimethylamino)propyl]methacrylamide (4.43 mL, 24.5 mmol) using substantially the same procedure as described in example above, except with a reduced reaction time of 15 h to afford cationic methacrylamide chloride functionalized tris(trimethylsiloxy)silane (14.2 g, 100%) as a colorless solid: ¹H NMR (CDCl3, 400 MHz) δ 9.06 (t, J=6 Hz, 1 H), 7.75 (t, J=6 Hz, 1 H), 5.85 (s, 1 H), 5.31 (s, 1 H), 4.40 (s, 2 H), 3.69-3.73-3.69 (m, 2 H), 3.45-3.38 (m, 2 H), 3.32 (s, 6 H), 3.18-3.13 (m, 2 H), 2.21-2.13 (m, 2 H), 1.93 (s, 3 H), 1.56-1.48 (m, 2 H), 0.42-0.37 (m, 2 H), 0.04 (s, 27 H); ESI-TOF MS data is summarized in Table 1.

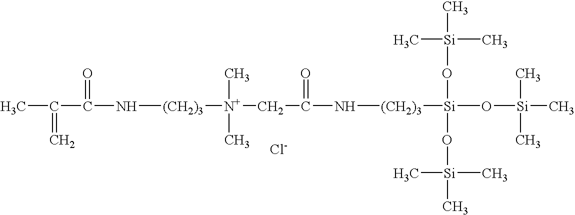

### Example 5

### Preparation of cationic methacrylate bromide Functionalized tris(trimethylsiloxy)silane

3-(Bromoacetylamido)propyltris(trimethylsiloxysilane) (10.1 g, 21.3 mmol) from Example 2 above was reacted with 2-(dimethylamino)ethyl methacrylate (3.76 mL, 22.3 mmol) using substantially the same procedure as described in example above to afford the product as a colorless, highly viscous liquid (13.9 g, 100%): ¹H NMR (CDCl₃, 400 MHz) δ 8.64 (t, J=5 Hz, 1 H), 6.10 (s, 1 H), 5.63 (s, 1 H), 4.72 (s, 2 H), 4.64 (br, 2 H), 4.20 (br, 2 H), 3.49 (s, 6 H), 3.20-3.15 (m, 2 H), 1.91 (s, 3 H), 1.58-1.50 (m, 2 H), 0.41 (t, J=8 Hz), 0.05 (s, 27 H); ESI-TOF MS data is summarized in Table 1.

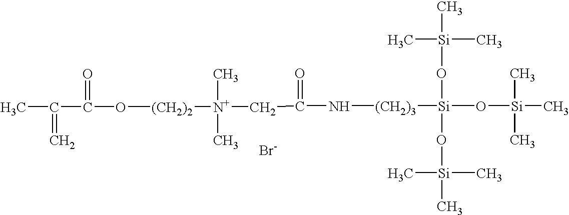

### Example 6

### Preparation of cationic methacrylamide bromide Functionalized tris(trimethylsiloxy)silane

3-(bromoacetylamido)propyltris(trimethylsiloxysilane) (10.0 g, 21.1 mmol) from Example 1 above was reacted with N-[3-(dimethylamino)propyl]methacrylamide (4.02 mL, 22.2 mmol) using substantially the same procedure as described in example 5 above to afford the product as a colorless, highly viscous liquid (14.1 g, 100%): ¹H NMR (CDCl₃, 400 MHz) δ 8.58 (t, J=6 Hz, 1 H), 7.42 (t, J=6 Hz, 1 H), 5.86 (s, 1 H), 5.33 (s, 1 H), 4.45 (s, 2 H), 3.75 (t, J=8 Hz, 2 H), 3.48-3.41 (m, 2 H), 3.35 (s, 6 H), 3.20-3.15 (m, 2 H), 2.23-2.13 (m, 2 H), 1.95 (s, 3 H), 1.57-1.49 (m, 2 H), 0.41 (t, J=8 Hz, 2 H), 0.05 (s, 27 H); ESI-TOF MS data is summarized in Table 1.

TABLE 1
ESI-TOF MS analysis of products from Examples 1–6

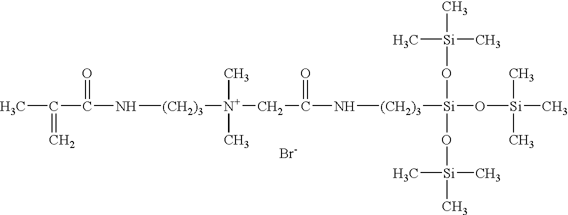

| | | Monoisotopic intact | Monoisotopic exact |
|---|---|---|---|
| Example | Elemental Composition | mass (Da) | mass error (ppm) |
| 1 | C₁₄H₃₆NO₄Si₄ClNa | 452.1332 | 6.6 |
| | | (Sodiated mass) | |
| 2 | C₁₄H₃₆NO₄Si₄BrNa | 496.0820 | 4.6 |
| | | (Sodiated mass) | |
| 3 | C₂₂H₅₁N₂O₆Si₄ | 551.2863 | 8.0 |
| 4 | C₂₃H₅₄N₃O₅Si₄ | 564.3174 | 6.9 |
| 5 | C₂₂H₅₁N₂O₆Si₄ | 551.2819 | 4.9 |
| 6 | C₂₃H₅₄N₃O₅Si₄ | 564.3159 | 4.3 |

### Examples 7-10

### Polymerization, Processing and Properties of Films Containing cationic siloxanyl monomers

Liquid monomer solutions containing cationic siloxanyl monomers from Examples 3-6 above, along with other additives common to ophthalmic materials (e.g., diluent, initiator, etc.) were clamped between silanized glass plates at various thicknesses and polymerized using thermal decomposition of the free-radical generating additive by heating 2 h at 100° C. under a nitrogen atmosphere. Each of the formulations listed in Table 2 afforded a transparent, tack-free, insoluble film.

TABLE 2
Formulations containing cationic siloxanyl monomers

| Ex. | Ex. 3 | Ex. 4 | Ex. 5 | Ex. 6 | NVP | HEMA | TRIS | PG | EGDMA | v-64 |
|---|---|---|---|---|---|---|---|---|---|---|
| 7 | 29.5 | | | | 21.6 | 21.7 | 22.2 | 4.3 | 0.2 | 0.5 |
| 8 | | 28.6 | | | 22.2 | 22.1 | 21.9 | 4.4 | 0.2 | 0.5 |
| 9 | | | 27.9 | | 32.9 | 33.0 | | 5.0 | 0.2 | 0.5 |
| 10 | | | | 28.3 | 32.7 | 32.9 | | 5.5 | 0.2 | 0.5 |

Films were removed from glass plates and hydrated/extracted in deionized H₂O for a minimum of 4 hours, transferred to fresh deionized H₂O and autoclaved 30 min at 121° C. The cooled films were then analyzed for selected properties of interest in ophthalmic materials as described in Table 3. Mechanical tests were conducted in borate buffered saline according to ASTM D-1708a, discussed above. The oxygen permeabilities, reported in Dk (or barrer) units, were measured in phosphate buffered saline at 35° C., using acceptable films with three different thicknesses, as discussed above.

TABLE 3
Properties of processed films containing cationic siloxanyl monomers

| | Water content | | Modulus | Tear |
|---|---|---|---|---|
| Example | (w/w %) | Dk (barrers) | (g/mm²)* | (g/mm)* |
| 7 | 70.0 | 47 | 69(6) | 23(1) |
| 8 | 64.7 | ND | 69(11) | 20(4) |
| 9 | 60.8 | 31 | 22(2) | 2.0(5) |
| 10 | 57.1 | 29 | 23(1) | 2.0(2) |

*number in parentheses indicates standard deviation of final digit(s)
ND = Not determined due to poor sample quality

### Example 11

### Preparation of 3-(chloroacetylamido)propyl Terminated poly(dimethylsiloxane)

To a vigorously stirred biphasic mixture of a solution of 3-aminopropyl terminated poly(dimethylsiloxane) (97.7 g, 3000 g/mol) obtained from Gelest, Inc., (Morrisville, Pa.) in dichloromethane (350 mL) and NaOH_{(aq)} (0.75 M, 150 mL) at 0° C. was added a solution of chloroacetyl chloride (8 mL, 0.1 mol) in dichloromethane (50 mL) dropwise. Following an additional 1 hour at ambient temperature, the organic layer was separated and stirred 5 hours over silica gel (25 g) and Na₂SO₄ (25 g) and filtered. Solvents were removed at reduced pressure to afford the product as a colorless liquid (85 g, 83%): ¹H NMR (CDCl₃, 400 MHz) δ 6.64 (br, 2 H), 4.05 (s, 4 H), 3.29 (q, J=7 Hz, 4 H), 1.60-1.52 (m, 4 H), 0.56-0.52 (m, 4 H), 0.06 (s, approximately 264 H); GPC: M_{w} 3075 g/mol, PD 1.80. The mass spectrum of this sample indicated a mass distribution of singly charged oligomers having a repeat unit mass of 74 Da. This corresponds to the targeted dimethyl siloxane (C₂H₆SiO) repeat unit chemistry. The targeted end group nominal mass for this sample is 326 Da (C₁₂H₂₄N₂O₂SiCl₂) and the required sodium charge agent has a mass of 23 Da (Na). The mass peaks in the distribution for this sample correspond to a nominal mass sequence of (74×n+326+23) where n is the number of repeat units. There is a good match between the experimental and theoretical isotopic distribution patterns for the oligomers evaluated.

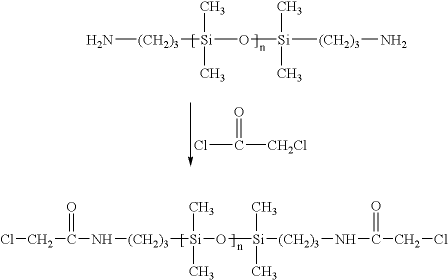

### Example 12

### Preparation of 3-(bromoacetylamido)propyl Terminated poly(dimethylsiloxane)

Aminopropyl terminated poly(dimethylsiloxane) (50.2 g, 3000 g/mol) was reacted with bromoacetyl chloride in substantially the same manner as described in Example 11 to afford the product as a viscous, colorless oil (40 g, 74%): ¹H NMR (CDCl₃, 400 MHz) δ 6.55 (br, 2 H), 3.89 (s, 4 H), 3.27 (q, J=7 Hz, 4 H), 1.60-1.52 (m, 4 H), 0.54 (t, J=7 Hz, 4 H), 0.06 (s, approximately 348 H). GPC: M_{w} 5762 g/mol, PD 1.77. The mass spectrum of this sample indicated a mass distribution of singly charged oligomers having a repeat unit mass of 74 Da. This corresponds to the targeted dimethyl siloxane (C2H6SiO) repeat unit chemistry. The targeted end group nominal mass for this sample is 414 Da (C₁₂H₂₄N₂O₂SiBr₂) and the required sodium charge agent has a mass of 23 Da (Na). The mass peaks in the distribution for this sample correspond to a nominal mass sequence of (74×n+414+23) where n is the number of repeat units. There is a good match between the experimental and theoretical isotopic distribution patterns for the oligomers evaluated.

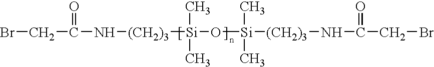

### Example 13

### Preparation of cationic methacrylate chloride Terminated poly(dimethylsiloxane)

To a solution of 3-(chloroacetylamido)propyl end-capped poly(dimethylsiloxane) (19.96 g) from Example 11 in ethyl acetate (25 mL) was added 2-(dimethylamino)ethyl methacrylate (3.40 mL, 20.1 mmol) and the mixture was heated 39 hours at 60° C. under a nitrogen atmosphere in the dark. The resulting solution was stripped of solvent and/or reagent at reduced pressure affording the product (23.1 g) containing a residual amount of 2-(dimethylamino)ethyl methacrylate (<10 w/w %) that is easily quantified by ¹H NMR analysis: ¹H NMR (CDCl₃, 400 MHz) δ 9.23 (br, 2 H), 6.07 (s, 2 H), 5.60 (s, 2 H), 4.71 (s, 4 H), 4.65-4.63 (m, 4 H), 4.18 (br, 4 H) 3.47 (s, 12 H), 3.19-3.13 (m, 4 H), 1.88 (s, 6 H), 1.53-1.49 (m, 4 H), 0.51-0.47 (m, 4 H), 0.01 (s, approximately 327 H). The mass spectrum of this sample indicated a mass distribution of doubly charged oligomers having a repeat unit mass of 37 Da. When deconvoluted this corresponds to a repeat unit mass of 74 Da (37 Da×2). This corresponds to the targeted dimethyl siloxane (C₂H₆SiO) repeat unit chemistry. The targeted end group nominal mass for this sample is 570 Da (C₂₈H₅₄N₄O₆Si). The end group chemistry contains two quaternary nitrogen atoms and thus no additional charge agent is required. The two quaternary nitrogen (N⁺) atoms also explain the presence of the doubly charged mass peaks. The mass peaks in the distribution for this sample correspond to a nominal mass sequence of ((74/2)×n+570) where n is the number of repeat units. There is a good match between the experimental and theoretical isotopic distribution patterns for the oligomers evaluated.

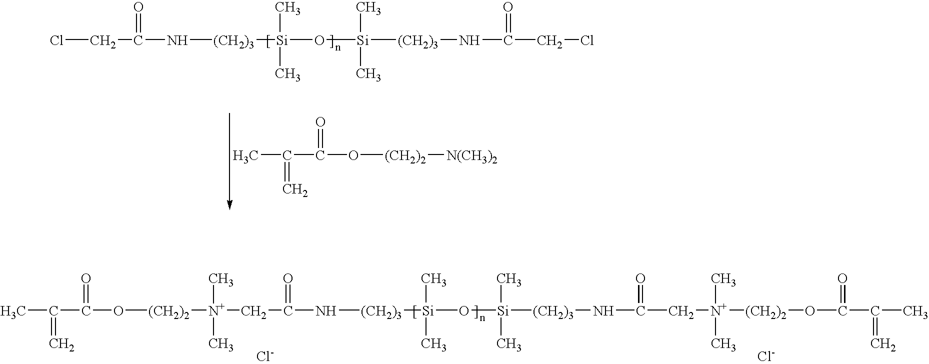

### Example 14

### Preparation of cationic methacrylamide chloride Terminated poly(dimethylsiloxane)

3-(Chloroacetylamido)propyl end-capped poly(dimethylsiloxane) from Example 11 (36.9 g) was reacted with N-[3-(dimethylamino)propyl]methacrylamide (4.90 mL, 27.0 mmol) in substantially the same fashion as described in Example 13 to afford cationic methacrylamide chloride terminated poly(dimethylsiloxane) (41.5 g) with a residual amount of N-[3-(dimethylamino)propyl]methacrylamide (<10 w/w %) that is easily quantified by ¹H NMR analysis: ¹H NMR (CDCl₃, 400 MHz) δ 9.19 (br, 2 H), 7.68 (br, 2 H), 5.87 (s, 2 H), 5.33 (br, 2 h), 4.45 (s, 4 H), 3.72-3.69 (m, 4 H), 3.44-3.40 (m, 4 H), 3.33 (s, 12 H), 3.21-3.16 (m, 4 H), 2.21-2.17 (m, 4 H), 1.95 (s, 6 H), 1.55-1.51 (m, 4 H), 0.54-0.49 (m, 4 H), 0.04 (s, approximately 312 H). The mass spectrum of this sample indicated a mass distribution of doubly charged oligomers having a repeat unit mass of 37 Da. When deconvoluted this corresponds to a repeat unit mass of 74 Da (37 Da×2). This corresponds to the targeted dimethyl siloxane (C₂H₆SiO) repeat unit chemistry. The targeted end group nominal mass for this sample is 596 Da (C₃₀H₆₀N₆O₄Si). The end group chemistry contains two quaternary nitrogen atoms and thus no additional charge agent is required. The two quaternary nitrogen (N⁺) atoms also explain the presence of the doubly charged mass peaks. The mass peaks in the distribution for this sample correspond to a nominal mass sequence of ((74/2)×n+596) where n is the number of repeat units. There is a good match between the experimental and theoretical isotopic distribution patterns for the oligomers evaluated.

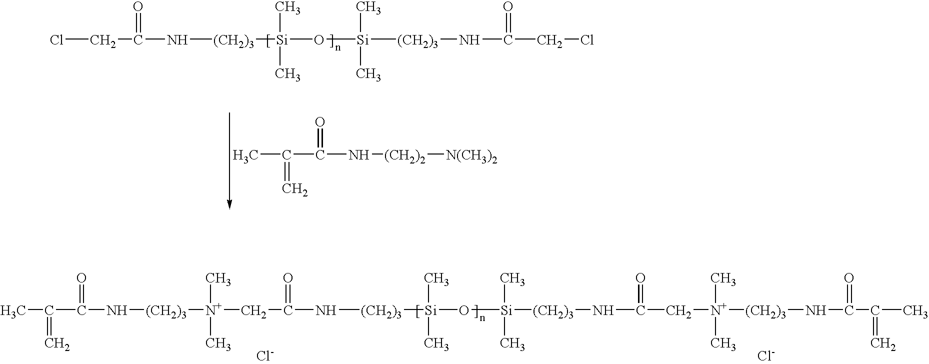

### Example 15

### Preparation of cationic methacrylate bromide Terminated poly(dimethylsiloxane)

3-(Bromoacetylamido)propyl terminated poly(dimethylsiloxane) from Example 12 (15.0 g) was reacted in substantially the same manner as described in Example 13 above to afford cationic methacrylate bromide terminated poly(dimethylsiloxane) (17.8 g) as a highly viscous liquid: ¹H NMR (CDCl₃, 400 MHz) δ 8.79 (br, 2 H), 6.12 (s, 2 H), 5.65 (s, 2 H), 4.76 (s, 4 H), 4.66 (br, 4 H), 4.20 (br, 4 H), 3.49 (s, 12 H), 3.21 (t, J=7 Hz, 4 H), 1.93 (s, 6 H), 1.59-1.51 (m, 4 H), 0.55-0.51 (m, 4 H), 0.04 (s, approximately 400 H). The mass spectrum of this sample indicated a mass distribution of doubly charged oligomers having a repeat unit mass of 37 Da. When deconvoluted this corresponds to a repeat unit mass of 74 Da (37 Da×2). This corresponds to the targeted dimethyl siloxane (C₂H₆SiO) repeat unit chemistry. The targeted end group nominal mass for this sample is 570 Da (C₂₈H₅₄N₄O₆Si). The end group chemistry contains two quaternary nitrogen atoms and thus no additional charge agent is required. The two quaternary nitrogen (N⁺) atoms also explain the presence of the doubly charged mass peaks. The mass peaks in the distribution for this sample correspond to a nominal mass sequence of ((74/2)×n+570) where n is the number of repeat units. There is a good match between the experimental and theoretical isotopic distribution patterns for the oligomers evaluated.

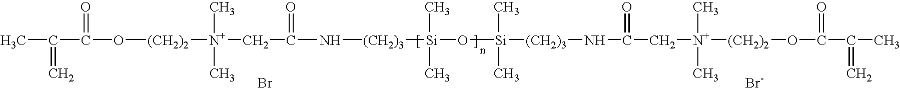

### Example 16

### Preparation of cationic methacrylamide bromide Terminated poly(dimethylsiloxane)

3-(Bromoacetylamido)propyl terminated poly(dimethylsiloxane) from Example 12 (15.0 g) was reacted in substantially the same manner as described in Example 13 above to afford cationic methacrylamide bromide terminated poly(dimethylsiloxane) as a highly viscous liquid (16.7 g): ¹H NMR (CDCl₃, 400 MHz) δ 8.76 (br, 2 H), 7.44 (br, 2 H), 5.87 (s, 2 H), 5.33 (s, 2 H), 4.47 (s, 4 H), 3.77-3.73 (m, 4 H), 3.43-3.40 (s, 4 H), 3.35 (s, 12 H), 3.22-3.17 (m, 4 H), 3.24-3.00 (m, 4 H), 1.96 (s, 6 H), 1.58-1.50 (m, 4 H), 0.54-0.50 (m, 4 H), 0.04 (s, approximately 387 H). The mass spectrum of this sample indicated a mass distribution of doubly charged oligomers having a repeat unit mass of 37 Da. When deconvoluted this corresponds to a repeat unit mass of 74 Da (37 Da×2). This corresponds to the targeted dimethyl siloxane (C₂H₆SiO) repeat unit chemistry. The targeted end group nominal mass for this sample is 596 Da (C₃₀H₆₀N₆O₄Si). The end group chemistry contains two quaternary nitrogen atoms and thus no additional charge agent is required. The two quaternary nitrogen (N⁺) atoms also explain the presence of the doubly charged mass peaks. The mass peaks in the distribution for this sample correspond to a nominal mass sequence of ((74/2)×n+596) where n is the number of repeat units. There is a good match between the experimental and theoretical isotopic distribution patterns for the oligomers evaluated.

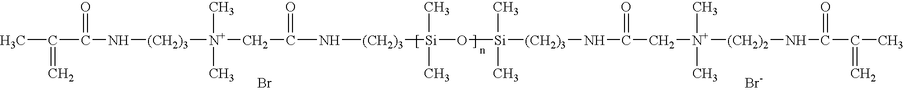

### Example 17

Preparation of cationic methacrylate chloride Terminated poly(dimethylsiloxane)

3-Aminopropyl terminated poly(dimethylsiloxane) (g, 900-1000 g/mol) was reacted in two steps in substantially the same manner as described in Examples 11 and 13 to afford cationic methacrylate chloride terminated poly(dimethylsiloxane) as a highly viscous fluid: ¹H NMR (CDCl₃, 400 MHz) δ 9.26 (br, 2 H), 6.12 (s, 2 H), 5.67 (s, 2 H), 4.75 (s, 4 H), 4.66 (br, 4 H), 4.14 (br, 4 H), 3.47 (s, 12 H), 3.22 (br, 4 H), 2.06 (br, 4 H), 1.93 (s, 6 H), 1.59-1.52 (m, 4 H), 0.56-0.52 (m, 4 H), 0.05 (s, approximately 192 H).

### Examples 18-23

Polymerization, processing and properties of films containing cationic siloxanyl prepolymers. Liquid monomer solutions containing cationic end-capped poly(dimethylsiloxane)prepolymers from Examples 13-17 above, along with other additives common to ophthalmic materials (diluent, initiator, etc.) were clamped between silanized glass plates at various thicknesses and polymerized using thermal decomposition of the free-radical generating additive by heating 2 hours at 100° C. under a nitrogen atmosphere. Each of the formulations listed in table 4 afforded a transparent, tack-free, insoluble film.

TABLE 4
Formulations containing cationic end-capped poly(dimethylsiloxane)

| Example | 13 | 14 | 15 | 16 | 17 | NVP | HEMA | TRIS | PG | EGDMA | v-64 |
|---|---|---|---|---|---|---|---|---|---|---|---|
| 18 | 19.2 | | | | | 34.4 | | 48.9 | | | 0.5 |
| 19 | 14.2 | | | | | 37.8 | 18.9 | 23.6 | 5.0 | | 0.5 |
| 20 | | 14.2 | | | | 37.9 | 19.0 | 23.7 | 4.7 | | 0.5 |
| 21 | | | 17.3 | | | 39.4 | 16.8 | 27.9 | 3.6 | 0.2 | 0.5 |
| 22 | | | | 25.8 | | 24.3 | 24.9 | 24.2 | | 0.2 | 0.5 |
| 23 | 7.0 | | | | 7.0 | 36.9 | 19.4 | 23.1 | 4.9 | | 0.5 |

Films were removed from glass plates and hydrated/extracted in deionized H₂O for a minimum of 4 hours, transferred to fresh deionized H₂O and autoclaved 30 min at 121° C. The cooled films were then analyzed for selected properties of interest in ophthalmic materials as described in table 5. Mechanical tests were conducted in borate buffered saline according to ASTM D-1708a, discussed above. The oxygen permeabilities, reported in Dk (or barrer) units, were measured in phosphate buffered saline at 35° C., using acceptable films with three different thicknesses, as discussed above.

TABLE 5
Properties of processed films containing cationic end-capped
poly(dimethylsiloxane)

| | Water content | Dk | Modulus | Tear |
|---|---|---|---|---|
| Example | (w/w %) | (barrers) | (g/mm²)* | (g/mm)* |
| 18 | 36.5 | 117 | 210(21) | 16(2) |
| 19 | 52.1 | 60 | 75(3) | 7.0(5) |
| 20 | 51.4 | 62 | 101(14) | 5(1) |
| 21 | 41.5 | 74 | 123(13) | 9(1) |
| 22 | 31.9 | 89 | 180(12) | 8.0(4) |
| 23 | 49.4 | 53 | 111(4) | 4.0(3) |

*number in parentheses indicates standard deviation of final digit(s)

### Example 24

Polymerization and processing of ophthalmic lenses containing cationic end-capped poly(dimethylsiloxane). 40 uL aliquots of a soluble, liquid monomer mix containing 13.9 parts by weight of the product from Example 13, 23.3 parts TRIS, 41.8 parts NVP, 13.9 parts HEMA, 5 parts PG, 0.5 parts v-64, 1.5 parts SA, and 60 ppm IMVT were sealed between poly(propylene) anterior and posterior contact lens moulds under an inert nitrogen atmosphere, transferred to an oven and heated under an inert nitrogen atmosphere 2 h at 100° C. The cooled mold pairs were separated and the dry lens released from the mold, hydrated/extracted twice in deionized H₂O for a minimum of 3 min, transferred to and sealed in an autoclave vial containing a buffered saline solution and autoclaved 30 min at 121° C. affording optically transparent, blue-tinted ophthalmic lenses with a refractive index of 1.4055±0.0005.

### Example 25

### Preparation of R-1778

Materials

The reagents bromobutene, 10% platinum-1,3-divinyl-1-1,1,3,3-tetramethyldisiloxane complex in xylenes, chloroform-d (99.8 atom % D), n-pentane (HPLC grade), anhydrous ehtyl acetate (99.8%), anhydrous tetrahydrofuran, anhydrous 1,4-dioxane, silica gel 60 (70-230 mesh ASTM) were purchased from Sigma-Aldrich, Milwaukee, Wis., and used without further purification. The reagent hydride terminated poly(dimethylsiloxane) (average molecular weight 1000-1100 g/mol) was purchased from Gelest, Inc. (Morrisville, Pa.).

Preparation

Step 1: Hydrosilation. To a solution of hydride terminated poly(dimethylsiloxane) (99.3 g, 1000-1100 Mₙ) and bromobutene (25 mL, 287 mmol, 3.0 eq.) in tetrahydrofuran/1,4-dioxane (2:1 v/v, 570 mL) in a round bottomed flask equipped with stirring apparatus, water condenser, and nitrogen purge was added 10% platinum-1,3-divinyl-1,1,3,3-tetramethyldisiloxane complex in xylenes (0.7 mL) and the solution was heated 4 h at 60° C. The cooled solution was concentrated under reduced pressure, redissolved in pentane (250 mL), passed through a chromatography column packed with silica gel (200 g) details in materials section and pentane, and flushed with an additional 300 mL pentane. The colorless solution was concentrated under reduced pressure (approximately 25 Torr), then stripped under high vacuum (approximately 1 Torr) to constant weight to afford 112.12 g (90.1% yield) clear liquid product (1316 g/mol).

Step 2: Quaternization. The colorless liquid product from step 1 (112.12 g) was then dissolved in ethyl acetate (150 mL, 1.3 mL/g) and treated with 2-(dimethylamino)ethyl methacrylate (116 mL, 680 mmol, approximately 8 eq.) in a round bottomed flask equipped with magnetic stir bar, and sealed with a nitrogen purge Torr a half hour. The reaction remained under positive nitrogen pressure upon removal of the nitrogen purge such that the vessel withstands the slight headspace pressure during subsequent heating. The reaction was then heated 100 h at 60° C. and in the dark. (NOTE: Due to the presence of polymerizable moiety, the reaction must be carefully monitored and controlled to avoid gelation, e.g. using a jacketed round bottom, oil bath, etc.). The cooled solution was then concentrated under reduced pressure (approximately 25 Torr and 40° C.). The resulting product mixture, ranging from viscous liquid to partial solid and clear to amber in color, was stripped at high vacuum (<1 Torr) and 60° C. to remove residual ethyl acetate and N,N-dimethylamino(ethyl methacrylate). The hot liquid will begin to solify into an amorphous solid during the stripped, requiring frequent stirring/scraping/crushing of the product mixture, especially toward the end of stripped. The stripping is complete when the product is solidified throughout by visual appearance and no more residual monomer is being collected. The resulting waxy solid product, ranging from colorless to light amber in color is then stored in amber vials at low temperature.

Analytical

¹H NMR: (CDCl₃, 400 MHz) δ 6.19 (s, 0.01 H), 5.66 (s, 0.01), 4.64 (br, 0.02 H), 1.76 (br, 0.02 H), 3.70-3.64 (m, 0.04 H), 3.50 (0.06 H), 1.94-1.83 (m, 0.05 H), 1.63-1.55 (m, 0.02 H), 0.05 (s, 0.78 H). PDMS chain length (×), molecular weight, percent conversion, and residual monomer/solvent are estimated using integrations of the product peaks at δ 5.66 (vinyl H of product end-cap, V), 5.55 (vinyl H of residual N,N-Dimethylamino(ethyl methacrylate)), 1.59 (CH₂ of PDMS alkyl end-cap, A), and 0.05 ppm (—CH₃ of PDMS backbone, P), using the following calculations:

Chain length (n)=(P×2)/(A×3)

Molecular weight (g/mol)=n×74+558

Conversion (%)=[(V×2)/(A)]×100

Mole fraction residual DMAEMA (d)=(D)/[(V/2)+(D)]

Residual DMAEMA (w/w %)=[(d×157)/([d×157]+[(1−d)×MW])]×100

ESI-TOF: The mass spectrum of this sample indicated a mass distribution of doubly charged oligomers having a repeat unit mass of 37 Da. When deconvoluted this corresponds to a repeat unit mass of 74 Da (37 Da×2). This corresponds to the targeted dimethyl siloxane (C₂H₆SiO) repeat unit chemistry. The end group chemistry contains two quaternary nitrogen atoms and thus no additional charge agent is required. The two quaternary nitrogen (N⁺) atoms also explain the presence of the doubly charged mass peaks.

### Example 26

### Preparation of RD-1799

Materials

The reagents chloroacetyl chloride (98%), 2-(dimethylamino)ethyl methacrylate (98%; IMPORTANT: Stabilized by 2000 ppm MEHQ), chloroform-d (99.8 atom % D), n-pentane (HPLC grade), anhydrous ethyl acetate (99.8%), sodium hydroxide, silica gel 60 (70-230 mesh ASTM) were purchased from Sigma-Aldrich, Milwaukee, Wis., and used without further purification. The reagent aminopropyl terminated poly(dimethylsiloxane) (average molcular weight 2500 g/mol) was purchased from Gelest, Inc. (Morrisville, Pa.).

Analytical Methods

ESI-TOF MS: The electrospray (ESI) time of flight (TOF) MS analysis was performed on an Applied Biosystems Mariner instrument. The instrument operated in positive ion mode. The instrument was mass calibrated with a standard solution containing lysine, angiotensinogen, bradykinin (fragment 1-5) and des-Pro bradykinin. This mixture provides a seven-point calibration from 147 to 921 m/z. The applied voltage parameters were optimized from signal obtained from the same standard solution. For exact mass measurements poly(ethylene glycol) (PEG), having a nominal Mₙ value of 400 Da, was added to the sample of interest and used as an internal mass standard. Two PEG oligomers that bracketed the sample mass of interest were used to calibrate the mass scale. Samples were prepared as 30 μM solutions in isopropanol (IPA) with the addition of 2% by volume saturated NaCl in IPA. Samples were directly infused into the ESI-TOF MS instrument at a rate of 35 μL/min. A sufficient resolving power (6000 RP m/Δm FWHM) was achieved in the analysis to obtain the monoistopic mass for each sample. In each analysis the experimental monoisotopic mass was compared to the theoretical monoisotopic mass as determined from the respective elemental compositions. In each analysis the monoisotopic mass comparison was less than 10 ppm error. It should be noted that uncharged samples have a sodium (Na) atom included in their elemental composition. This Na atom occurs as a necessary charge agent added in the sample preparation procedure. Some samples do not require an added charge agent since they contain a charge from the quaternary nitrogen inherent to their respective structure.

NMR: ¹H-NMR characterization was carried out using a 400 MHz Varian spectrometer using standard techniques in the art. Samples were dissolved in chloroform-d (99.8 atom % D), unless otherwise noted. Chemical shifts were determined by assigning the residual chloroform peak at 7.25 ppm. Peak areas and proton ratios were determined by integration of baseline separated peaks. Splitting patterns (s=singlet, d=doublet, t=triplet, q=quartet, m=multiplet, br=broad) and coupling constants (J/Hz) are reported when present and clearly distinguishable.

SEC: Size Exclusion Chromatography (SEC) analyses were carried out by injection of 100 μL of sample dissolved in tetrahydrofuran (THF) (5-20 mg/mL) onto a Polymer Labs PL Gel Mixed Bed E (×2) column at 35° C. using a Waters 515 HPLC pump and HPLC grade THR mobile phase flow rate of 1.0 mL/min, and detected by a Waters 410 Differential Refractometer at 35° C. Values of Mₙ and M_{w} and polydispersity (PD) were determined by comparison to Polymer Lab Polystyrene narrow standards.

Preparation

Step 1: Amidation. To a vigorously stirred biphasic mixture of 3-aminopropyl terminated poly(dimethylsiloxane) (97.7 g) in dichloromethane (122 mL) and NaOH(_{aq}) (5.0 M, 62 mL) at 0° C. was added a solution of chloroacetyl chloride (9.31 mL, 0.117 mol) in dichloromethane (23 mL) dropwise over 30 min. Following an additional 1.5 h at 0° C., the organic layer was separated and dried over magnesium sulfate. The clear liquid was decanted and passed through a chromatography column packed with silica gel (150 g) and methylene chloride. An additional 200 mL of methylene chloride was passed through the column and solvents were removed at reduced pressure to afford the product as a viscous, colorless liquid (85 g, 83%).

¹H NMR: (CDCl₃, 400 MHz) δ 6.64 (br, 2 H), 4.05 (s, 4 H), 3.29 (q, J=7 Hz, 4 H), 1.60-1.52 (m, 4 H), 0.56-0.52 (m, 4 H), 0.06 (s, approximately 264 H).

SEC: M_{w} 3075 g/mol, PD 1.80.

ESI-TOF: The mass spectrum of this sample indicated a mass distribution of singly charged oligomers having a repeat unit mass of 74 Da. This corresponds to the targeted dimethyl siloxane (C2H6SiO) repeat unit chemistry. The targeted end group nominal mass for this sample is 326 Da (C₁₂H₂₄N₂O₂SiCl₂) and the required sodium charge agent has a mass of 23 Da (Na). The mass peaks in the distribution for this sample correspond to a nominal mass sequence of (74×n+326+23) where n is the number of repeat units. There is a good match between the experimental and theoretical isotopic distribution patterns for the oligomers evaluated.

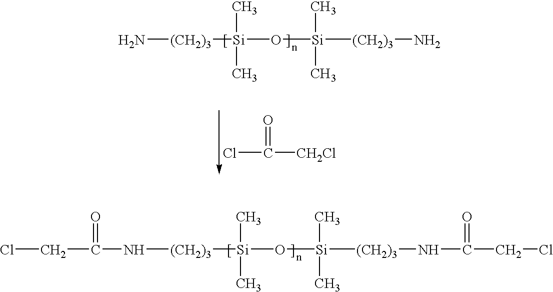

Step 2: Quaternization. A solution of 3-(chloroacetylamido)propyl end-capped poly(dimethylsiloxane) (19.96 g, 3200 g/mol) from step 1, ethyl acetate (19 mL), and para-methoxyphenol (20 mg, 1000 ppm) in a round-bottomed flask equipped with stir bar, was treated with 2.25 equivalents 2-(dimethylamino)ethyl methacrylate. To account for slight differences in molecular weight distributions, 2-(dimethylamino)ethyl methacrylate was added in small quantities, homogenized with stirring, then aliquots of reaction mixture were removed, diluted in chloroform-d and analyzed via ¹H NMR integration of the multiplet peak at 0.56-0.52 ppm (4 protons per end-capped PDMS) versus the singlet peak at 5.55 ppm (1 proton per 2-(dimethylamino)ethyl methacrylate) to obtain accurate quantification of stoichiometry, then adjusted as needed with additional 2-(dimethylamino)ethyl methacrylate. The vessel was sealed and purged with nitrogen 30 min. The purge was removed and positive nitrogen pressure remained such that the vessel withstands the slight headspace pressure during subsequent heating. The reaction was then heated 80 h at 60° C. and in the dark. (NOTE: Due to the presence of polymerizable moiety, the reaction must be carefully monitored and controlled to avoid gelation, e.g., using a jacketed round bottom, oil bath, etc.). The cooled solution was then concentrated under reduced pressure (approximately 25 Torr and 40° C.), then stripped at high vacuum (<1 Torr) and ambient temperature to constant weight (4-15 h) affording the product as a highly viscous liquid ranging from colorless to yellow containing a residual amount or 2-(dimethylamino)ethyl methacrylate (<10 w/w %) that is then transferred into amber bottles and stored cold.

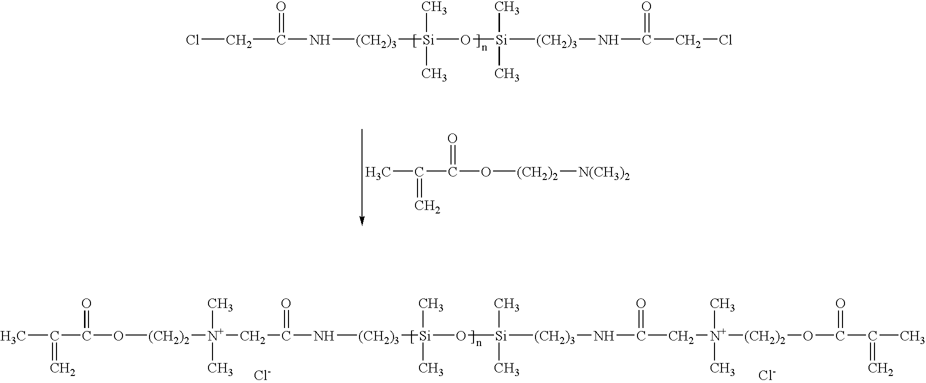

Analytical

¹H NMR: (CDCl₃, 400 MHz) δ 9.23 (br, 2 H), 6.07 (s, 2 H), 5.60 (s, 2 H), 4.71 (s, 4 H), 4.65-4.63 (m, 4 H), 4.18 (br, 4 H) 3.47 (s, 12 H), 3.19-3.13 (m, 4 H), 1.88 (s, 6 H), 1.53-1.49 (m, 4 H), 0.51-0.47 (m, 4 H), 0.01 (s, approximately 327 H). PDMS chain length (×), molecular weight, percent conversion, and residual monomer/solvent are estimated using integrations of the product peaks at δ 5.60 (vinyl H of product end-cap, V), 5.55 (vinyl H of residual N,N-Dimethylamino(ethyl methacrylate)), 0.51-0.47 (CH₂ of PDMS alkyl end-cap, A), and 0.01 ppm (—CH₃ of PDMS backbone, P), using the following calculations:

Chain length (n)=(P×2)/(A×3)

Molecular weight (g/mol)=n×74+584

Conversion (%)=[(V×2)/(A)]×100

Mole fraction residual DMAEMA (d)=(D)/[V/2)+(D)]

Residual DMAEMA (w/w %)=[(d×157)/([d×157]+[(1−d)×MW])]×100

ESI-TOF: The mass spectrum of this sample indicated a mass distribution of doubly charged oligomers having a repeat unit mass of 37 Da. When deconvoluted this corresponds to a repeat unit mass of 74 Da (37 Da×2). This corresponds to the targeted dimethyl siloxane (C₂H₆SiO) repeat unit chemistry. The targeted end group nominal mass for this sample is 570 Da (C₂₈H₅₄N₄O₆Si). The end group chemistry contains two quaternary nitrogen atoms and thus no additional charge agent is required. The two quaternary nitrogen (N⁺) atoms also explain the presence of the doubly charged mass peaks. The mass peaks in the distribution for this sample correspond to a nominal mass sequence of ((74/2)×n+570) where n is the number of repeat units. There is a good match between the experimental and theoretical isotopic distribution patterns for the oligomers evaluated.

### Example 27

### Preparation of RD-1799-B and RD-1778-B

Materials

The reagents 2-(dimethylamino)ethyl methacrylate (98%; IMPORTANT: Stabilized by 2000 ppm MEHQ), trifluoroacetic acid, chloroform-d (99.8 atom % D), n-pentane (HPLC grade), anhydrous ethyl acetate (99.8%), sodium hydroxide, silica gel 60 (70-230 mesh ASTM) were purchased from Sigma-Aldrich (Milwaukee, Wis.), and used without further purification. The reagent Octamethylcyclotetrasiloxane (D₄), was purchased from Gelest, Inc. (Morrisville, Pa.), and the reagent 1,3-bis(4-bromobutyl)tetramethyldisiloxane was purchased from Silar Laboratories (Scotia, N.Y.).

Analytical Methods

ESI-TOF MS: The electrospray (ESI) time of flight (TOF) MS analysis was performed on an Applied Biosystems Mariner instrument. The instrument operated in positive ion mode. The instrument was mass calibrated with a standard solution containing lysine, angiotensinogen, bradykinin (fragment 1-5) and des-Pro bradykinin. This mixture provides a seven-point calibration from 147 to 921 m/z. The applied voltage parameters were optimized from signal obtained from the same standard solution. For exact mass measurements poly(ethylene glycol) (PEG), having a nominal Mₙ value of 400 Da, was added to the sample of interest and used as an internal mass standard. Two PEG oligomers that bracketed the sample mass of interest were used to calibrate the mass scale. Samples were prepared as 30 μM solutions in isopropanol (IPA) with the addition of 2% by volume saturated NaCl in IPA. Samples were directly infused into the ESI-TOF MS instrument at a rate of 35 L/min. A sufficient resolving power (6000 RP m/Δm FWHM) was achieved in the analysis to obtain the monoistopic mass for each sample. In each analysis the experimental monoisotopic mass was compared to the theoretical monoisotopic mass as determined from the respective elemental compositions. In each analysis the monoisotopic mass comparison was less than 10 ppm error. It should be noted that uncharged samples have a sodium (Na) atom included in their elemental composition. This Na atom occurs as a necessary charge agent added in the sample preparation procedure. Some samples do not require an added charge agent since they contain a charge from the quaternary nitrogen inherent to their respective structure.

NMR: ¹H-NMR characterization was carried out using a 400 MHz Varian spectrometer using standard techniques in the art. Samples were dissolved in chloroform-d (99.8 atom % D), unless otherwise noted. Chemical shifts were determined by assigning the residual chloroform peak at 7.25 ppm. Peak areas and proton ratios were determined by integration of baseline separated peaks. Splitting patterns (s=singlet, d=doublet, t=triplet, q=quartet, m=multiplet, br=broad) and coupling constants (J/Hz) are reported when present and clearly distinguishable.

SEC: Size Exclusion Chromatography (SEC) analyses were carried out by injection of 100 μL of sample dissolved in tetrahydrofuran (THF) (5-20 mg/mL) onto a Polymer Labs PL Gel Mixed Bed E (×2) column at 35° C. using a Waters 515 HPLC pump and HPLC grade THF mobile phase flow rate of 1.0 mL/min, and detected by a Waters 410 Differential Refractometer at 35° C. Values of Mₙ, M_{w}, and polydispersity (PD) were determined by comparison to Polymer Lab Polystyrene narrow standards.

Preparation

Step 1: Ring-Opening Polymerization. A solution of 1,3-bis(4-bromobutyl)tetramethyldisiloxane and octamethylcyclotetrasiloxane in a flask equipped with a stir bar and drying column was treated with trifluororoacetic acid and stirred 24 h at ambient T. To the reaction was added NaHCO₃ and the mixture was allowed to stir an additional 24 h at ambient T. The mixture was then filtered with pressure through a 5 μm PTFE filter, then stripped 2 h at 80° C. and 1-5 Torr to afford the product as a transparent, colorless, viscous liquid.

Step 2: Quaternization. The colorless liquid product from step 1 was then dissolved in ethyl acetate and treated with 2-(dimethylamino)ethyl methacrylate in a round bottomed flask equipped with magnetic stir bar. The reaction vessel was sealed in a manner to withstand the slight headspace pressure during subsequent heating. The reaction was then heated 100 h at 60° C. and in the dark. (NOTE: Due to the presence of polymerizable moiety, the reaction must be carefully monitored and controlled to avoid gelation, e.g., using a jacketed round bottom, oil bath, etc.). The cooled solution was then concentrated under reduced pressure (approximately 25 Torr and 40° C.). The resulting product mixture, ranging from viscous liquid to partial solid and clear to amber in color, was stripped at high vacuum (<1 Torr) and 60° C. to remove residual ethyl acetate and N,N-dimethylamino(ethyl methacrylate). Due to partial solidification during the stripping, frequent stirring/scraping/crushing of the product mixture, especially toward the end of stripping and especially for M₂D₁₄plus-B, may be required. The stripping is complete when no more residual monomer is being collected, and should not exceed 8 h. The resulting waxy solid product, ranging from colorless to light amber in color is then stored in amber vials at low temperature.

### Example 28

### Preparation of a Cationic Contact Lens

A monomer mixture was prepared by mixing the following components: M₂D₃₉, a monomer of formula (XI) where n is about 39; n-vinyl-2-pyrrolidone (NVP); tris(trimethylsiloxy)silylpropyl methacrylate (Tris); 2-hydroxyethylmethacrylate (Hema); a diluent, propylene glycol; a UV blocker, 2-(3-(2H-benzotriazol-yl)-4-hydroxy-phenyl)ethyl methacrylate; Vaso-64 initiator; and tint agent, 1,4-bis[4-(2-methacryloxyethyl)phenylamino]anthraquinone. The mixture was added to a two-part polypropylene mold, including a posterior mold half for forming the posterior contact lens surface, and an anterior mold half for forming the anterior mold half. The mixture was cured thermally while contained in the mold. The resultant contact lenses were removed from the mold, extracted and hydrated.

### Example 29A-C

### Preparation of Packaging Solutions

Three packaging solution compositions within the scope of the present invention were prepared by mixing an anionic polyacrylic acid with a buffer solution prepared from 1.0% boric acid, 0.11% sodium borate and 0.40% NaCl. The anionic polyacrylic acids used in each example are set forth below in Table 6

TABLE 6

| Example | Polyacrylic Acid |
|---|---|
| EX. 29A | 1% P(AA) Na⁺, M_{w} = 6,000 Da |
| EX. 29B | 1% P(AA) Na⁺, M_{w} = 60,000 Da |
| EX. 29C | 1% P(AA) Na⁺, M_{w} = 225,000 Da |

Testing

The solutions containing the respective anionic polyacrylic acids in Table 6 were tested as follows. The contact lenses of Example 28 were soaked in the respective solutions containing the anionic polyacrylic acids of Table 6 for no fewer than 72 hours. The lenses were then removed from the test solution and were immediately mounted and tested in 1 mL phosphate borate saline (PBS). Tribological testing was performed on a CETR Model UMT-2 micro-tribometer. Tribology is the study of how two surfaces interact with each other when in relative motion. One aspect of tribology that may be of importance to contact lenses is friction. Friction is a measure of a material's resistance to lateral motion when placed against a specific substrate. The relative friction between two surfaces may be described in terms of a coefficient of friction (COF), which is defined as the ratio of the lateral force (Fₓ) that is required to initiate and then sustain movement to the normal force (F_{N}). Further, there are two friction coefficients that may be considered, the peak (or static) and average (or kinetic). The static COF is a measure of how much Fₓ is needed to initiate relative motion of two surfaces and is typically the larger of the two values. Practically, for contact lenses, the static COF is related to the amount of force needed to start a blink cycle or for the lens to begin moving over the cornea. The kinetic COF is a measure of how much lateral force is needed to sustain movement at a particular velocity averaged over a finite period of time. This value is related to the amount of force required to sustain the blink over the course of the entire cycle and the ease of motion of the lens on the cornea (which may be further related to how much the lens moves on the cornea).

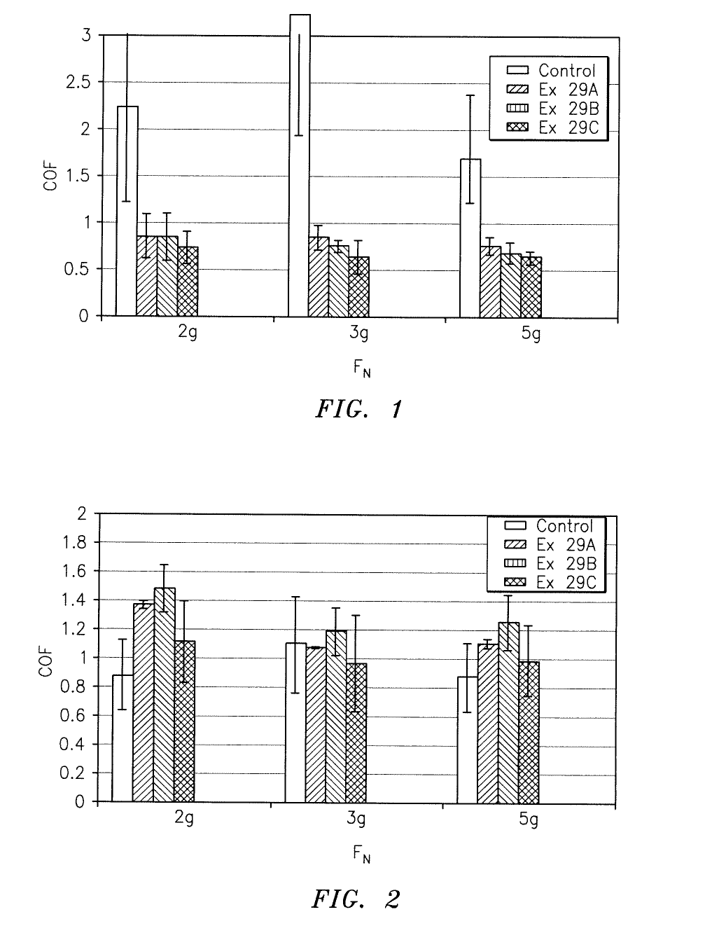

Each lens was clamped on an HDPE holder that initially mates with the posterior side of the lens. A poly(propylene) clamping ring was then used to hold the edge region of the lens. Once the lens was mounted in the holder the assembly was placed in a stationary clamping device within the micro-tribometer. A polished stainless steel disc containing 1 mL of the test solution was then brought into contact with the lens and F_{N} was adjusted to 2 grams over the course of the run for the frictional measurements. Additional runs were made with F_{N} adjusted to 3 grams and 5 grams. After the load equilibrated for 5 seconds the stainless steel disc was rotated at a velocity of 12 cm/sec for a duration of 20 seconds in both the forward and reverse directions and the peak (static) and average (kinetic) COF values were recorded. Each value represents the average of 4-5 lenses. Controls were run from the same lot of lenses that were soaked in a control buffer solution containing only 1.0% boric acid, 0.11% sodium borate and 0.40% NaCl for no fewer than 72 hours. All data was normalized to the average values obtained from the HDPE holder in 1 mL phosphate buffered saline in the absence of a contact lens. The results are summarized in FIGS. 1 and 2.

FIG. 1 shows the results of the static COF values determined at the three normal loads for the contact lenses in each of the packaging solutions, where the error bars represent the standard deviation. The static COF value is a measure of the lateral force required to initiate the movement of the stainless steel disk from a resting position. Once the disk is in motion, there is a certain amount of lateral force that needs to be applied to overcome the interactive forces between the lens surface and the disk in order to maintain the desired velocity. The force required to maintain movement will be significantly less than the force required to initiate movement and will be used to determine the kinetic COF values as presented in FIG. 2. The reported kinetic COF value for each solution represents an average value over the 20 seconds duration of the test.

In general, all of the test solutions were effective at reducing the static COF value while increasing the kinetic COF.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A method of preparing a package comprising a storable, sterile cationic ophthalmic device, the method comprising:
(a) immersing an ophthalmic device having at least one cationic surface in a solution comprising an effective amount of a soluble anionic polymer, wherein the solution has an osmolality of at least about 200 mOsm/kg and a pH in the range of about 6 to about 9;
(b) packaging the solution and the device in a manner preventing contamination of the lens by microorganisms; and
(c) sterilizing the packaged solution and device.

2. The method of claim 1, wherein the cationic ophthalmic device is a cationic ophthalmic lens.

3. The method of claim 1, wherein the cationic ophthalmic device is a cationic contact lens.

4. The method of claim 1, wherein the cationic ophthalmic device comprises a polymerization product of a monomeric mixture comprising one or more cationic silicone-containing monomers.

5. The method of claim 1, wherein the cationic ophthalmic device comprises a polymerization product of a monomeric mixture comprising one or more one or more cationic silicone-containing monomers and one or more hydrophilic monomers.

6. The method of claim 1, wherein the cationic ophthalmic device comprises a polymerization product of a monomeric mixture comprising one or more cationic silicon-containing monomers of formula I: 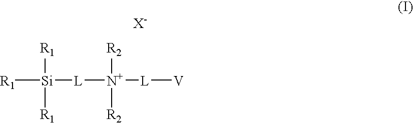
wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; R₁ and R₂ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group and V is independently a polymerizable ethylenically unsaturated organic radical.

7. The method of claim 1, wherein the cationic ophthalmic device comprises a polymerization product of a monomeric mixture comprising one or more cationic silicon-containing monomers of formula II: 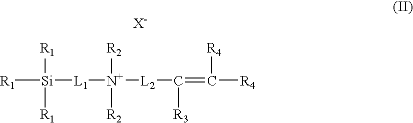
wherein each R₁ is the same and is —OSi(CH₃)₃ R₂ is methyl, L₁ is an alkyl amide, L₂ is a alkyl amide or ester having 2 or 3 carbon atoms that is joined to a polymerizable vinyl group, R₃ is methyl, R₄ is H and X⁻ is Br⁻ or Cl⁻.

8. The method of claim 1, wherein the cationic ophthalmic device comprises a polymerization product of a monomeric mixture comprising one or more cationic silicon-containing monomers of formula VIII: 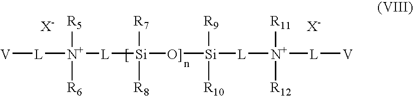
wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; R₅, R₆, R₇, R₈, R₉, R₁₀, R₁₁ and R₁₂ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; V is independently a polymerizable ethylenically unsaturated organic radical and n is an integer of 1 to about 300.

9. The method of claim 1, wherein the cationic ophthalmic device comprises a polymerization product of a monomeric mixture comprising one or more cationic silicon-containing monomers of formula XIV: 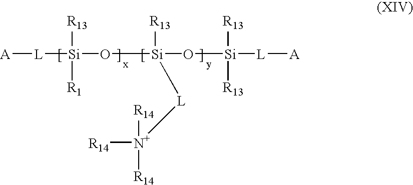
wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; each R₁, R₁₃ and R₁₄ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; A is a polymerizable vinyl moiety; x is 0 to 1000, and y is 1 to 300.

10. The method of claim 1, wherein the cationic ophthalmic device comprises a polymerization product of a monomeric mixture comprising one or more cationic random copolymers of formula XVI: 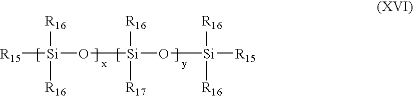
wherein x is 0 to 1000, y is 1 to 300; each R₁₅ and R₁₆ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; R₁₇ is independently one or more of the following formulae XVII and XVIII: 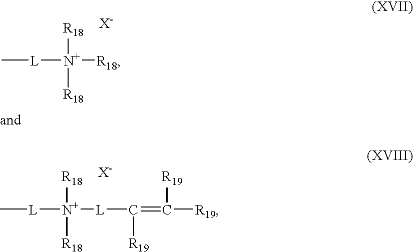
wherein L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; R₁₈ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; and R₁₉ is independently hydrogen or methyl

11. The method of claim 1, wherein the soluble anionic polymer in the solution is selected from the group consisting of an anionic cellulose-containing polymer, an anionic polymer derived from acrylic acid and esters thereof, an anionic polymer derived from methacrylic acid and esters thereof and mixtures thereof.

12. The method of claim 1, wherein the soluble anionic polymer in the solution is selected from the group consisting of polyacrylic acid, polymethacrylic acid, polyethylene amine, polysaccharide, hyaluronic acid, chondroitin sulfate and mixtures thereof.

13. The method of claim 1, wherein the soluble anionic polymer is present in the solution in an amount of about 0.05% w/v to about 5% w/v.

14. The method of claim 1, wherein the solution further comprises a buffering agent.

15. The method of claim 1, further comprising hermetically sealing the ophthalmic device and the packaging solution in the package.

16. The method of claim 15, wherein heat sterilization is performed subsequent to sealing of the package.

17. The method of claim 1, wherein the solution does not contain an effective disinfecting amount of a disinfecting agent.

18. The method of claim 1, wherein the solution does not contain a germicide compound.

19. A packaging system for the storage of a cationic ophthalmic device comprising a sealed container containing one or more unused cationic ophthalmic devices immersed in an aqueous packaging solution comprising an effective amount of a soluble anionic polymer, wherein the solution has an osmolality of at least about 200 mOsm/kg, a pH of about 6 to about 9 and is heat sterilized.

20. The system of claim 19, wherein the cationic ophthalmic device is a cationic ophthalmic lens.

21. The system of claim 19, wherein the cationic ophthalmic device is a cationic contact lens.

22. The system of claim 19, wherein the cationic ophthalmic device comprises a polymerization product of a monomeric mixture comprising one or more cationic silicone-containing monomers.

23. The system of claim 19, wherein the cationic ophthalmic device comprises a polymerization product of a monomeric mixture comprising one or more cationic silicon-containing monomers and one or more hydrophilic monomers.

24. The system of claim 19, wherein the cationic ophthalmic device comprises a polymerization product of a monomeric mixture comprising one or more cationic silicon-containing monomers of formula I: 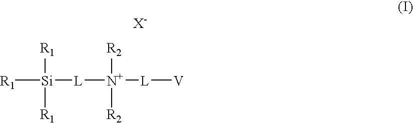
wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; R₁ and R₂ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group and V is independently a polymerizable ethylenically unsaturated organic radical.

25. The system of claim 19, wherein the cationic ophthalmic device comprises a polymerization product of a monomeric mixture comprising one or more cationic silicon-containing monomers of formula II: 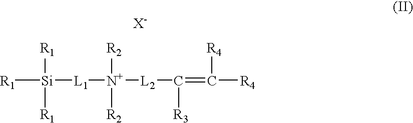
wherein each R₁ is the same and is —OSi(CH₃)₃, R₂ is methyl, L₁ is an alkyl amide, L₂ is a alkyl amide or ester having 2 or 3 carbon atoms that is joined to a polymerizable vinyl group, R₃ is methyl, R₄ is H and X⁻ is Br⁻ or Cl⁻.

26. The system of claim 19, wherein the cationic ophthalmic device comprises a polymerization product of a monomeric mixture comprising one or more cationic silicon-containing monomers of formula VIII: 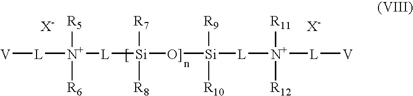
wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; R₅, R₆, R₇, R₈, R₉, R₁₀, R₁₁ and R₁₂ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; V is independently a polymerizable ethylenically unsaturated organic radical and n is an integer of 1 to about 300.

27. The system of claim 19, wherein the cationic ophthalmic device comprises a polymerization product of a monomeric mixture comprising one or more cationic silicon-containing monomers of formula XIV: 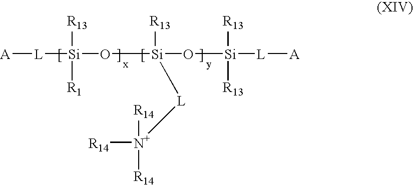
wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; each R₁, R₁₃ and R₁₄ are each independently hydrogen, a straight or branched C₁-C₃o alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; A is a polymerizable vinyl moiety; x is 0 to 1000, and y is 1 to 300.

28. The system of claim 19, wherein the cationic ophthalmic device comprises a polymerization product of a monomeric mixture comprising one or more cationic random copolymers of formula XVI: 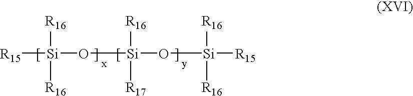
wherein x is 0 to 1000, y is 1 to 300; each R₁₅ and R₁₆ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; R₁₇ is independently one or more of the following formulae XVII and XVIII: 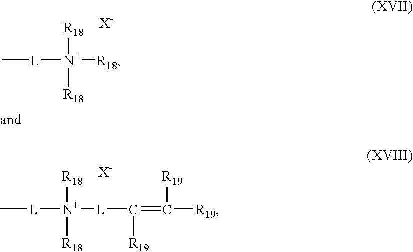
wherein L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof; X⁻ is at least a single charged counter ion; R₁₈ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group; and R₁₉ is independently hydrogen or methyl

29. The system of claim 19, wherein the soluble anionic polymer in the solution is selected from the group consisting of an anionic cellulose-containing polymer, an anionic polymer derived from acrylic acid and esters thereof, an anionic polymer derived from methacrylic acid and esters thereof and mixtures thereof.

30. The system of claim 19, wherein the soluble anionic polymer in the solution is selected from the group consisting of polyacrylic acid, polymethacrylic acid, polyethylene amine, polysaccharides, hyaluronic acid, chondroitin sulfate and mixtures thereof.

31. The system of claim 19, wherein the soluble anionic polymer is present in the solution in an amount of about 0.05% w/v to about 5% w/v.

32. The system of claim 19, wherein package is heat sterilized subsequent to sealing of the package.

33. The system of claim 19, wherein the solution does not contain an effective disinfecting amount of a disinfecting agent.

34. The system of claim 19, wherein the solution does not contain a germicide compound.
